(19) United States
(12) Patent Application Publication
(10) Pub. No.: US 2026/0165887 A1
(43) Pub. Date: Jun. 18, 2026

(54) ZONAL TOPSHEET FOR AN ABSORBENT ARTICLE

(71) Applicant: The Procter & Gamble Company, Cincinnati, OH (US)

(72) Inventors: Adrien GRENIER, Schwalbach (DE); Gueltekin ERDEM, Cincinnati, OH (US)

(21) Appl. No.: 19/419,432

(22) Filed: Dec. 15, 2025

Related U.S. Application Data
(60) Provisional application No. 63/733,711, filed on Dec. 13, 2024.
(60) Provisional application No. 63/784,324, filed on Apr. 7, 2025.

(51) Int. Cl. A61F 13/49; A61F 13/15; A61F 13/511; A61F 13/512; A61F 13/513; A61F 13/514

(52) U.S. Cl. CPC A61F 13/49; A61F 13/15707; A61F 13/51104; A61F 13/51113; A61F 13/5126; A61F 13/51305; A61F 13/514

(57) ABSTRACT

An absorbent article is provided including a first and second end edge and a longitudinal length L between the first and second end edge. The absorbent article also includes a liquid permeable topsheet, a liquid impermeable backsheet, and an absorbent core disposed between the topsheet and the backsheet. The topsheet comprises at least two visually discernible zones comprising a first zone and a second zone. The first zone has a first zone length that is 0.5 L to L, where the first zone has a minimum width along the first zone length that is in the range of 0.7 W to 1.3 W. The first zone has a different core height value and/or a different core void volume, according to the Surface Topography Test. The first and second zones each comprise apertures and/or three-dimensional features. The three-dimensional features comprise morphological features that are at least partially concave or convex.

### CROSS REFERENCE TO RELATED APPLICATION

This application claims the benefit of U.S. Provisional Patent Application Nos. 63/733,711, filed on Dec. 13, 2024, and 63/784,324, filed on Apr. 7, 2025, the entire disclosure of each of which is incorporated herein by reference.

### FIELD

The present disclosure relates generally to absorbent articles and more specifically to a zonal topsheet for an absorbent article.

### BACKGROUND

Absorbent articles, such as diapers, have featured zonal topsheets which include one or more visually discernable zones. For example, as shown in FIG. 14, a zonal topsheet 26′ has been provided for absorbent articles and includes multiple absorbent zones 192, 194 along a central longitudinal axis 50 extending from the front end edge 18 to the rear end edge 20 of the topsheet 26′. Additionally, another zone 196 is provided that surrounds the multiple absorbent zones 192, 194. The zone 192 is known as a “liquid excrement zone” and the zone 194 is known as a “solid excrement zone” and thus the topsheet 26′ is designed with the intention of placing the liquid excrement zone 192 and the solid excrement zone 194 at different locations along the longitudinal axis 50 where liquid excrement and solid excrement, respectively, are expected to end up when the absorbent article is worn.

However, topsheets for absorbent articles, such as diapers have several drawbacks. For example, the zonal topsheet 26′ is provided with the liquid excrement zone 192 and solid excrement zone 194 in locations where liquid excrement and solid excrement, respectively, are expected to be discharged from the wearer. However, it was determined from consumers that in practice this is not the case. Instead, consumers repeatedly reported that the liquid excrement and solid excrement discharged from the wearer ends up all over the topsheet 26′ and thus the liquid excrement and solid excrement are not typically confined to the liquid excrement and solid excrement zones 192, 194, respectively.

The discussion of shortcomings and needs existing in the field prior to the present disclosure is in no way an admission that such shortcomings and needs were recognized by those skilled in the art prior to the present disclosure.

### SUMMARY

Various embodiments solve the above-mentioned problems and provide methods and devices useful for providing a zonal topsheet for an absorbent article with multiple visually discernable zones including an absorbent zone along the central longitudinal axis 50 which encompasses those regions of the topsheet where discharged liquid excrement and solid excrement are expected to be located. The improved zonal topsheet herein is designed in response to consumers who reported that the liquid excrement and solid excrement zones 192, 194 of the topsheet 26′ do not work in practice since liquid excrement is not typically confined to the liquid excrement zone 192 and solid excrement is not typically confined to the solid excrement zone 194. Instead, the consumers report that the liquid excrement and solid excrement substantially overlap along the topsheet and are positioned over a wide portion of the central longitudinal axis 50. Thus the zonal topsheet with the spaced apart liquid excrement and solid excrement zones 192, 194 compromises performance since it unnecessarily reduces a range of absorption along the central longitudinal axis 50 (since no absorbent zone is provided between the zones 192, 194 where liquid excrement and solid excrement can end up). Thus, the improved zonal topsheet herein may disclose a single central absorbent zone along the central longitudinal axis 50 which is positioned in a region where liquid excrement and solid excrement are expected along the topsheet.

Additionally, consumers reported that it is desired to have additional comfort zones around the central absorbent zone which have different features (e.g. softer, smaller apertures, smoother, etc.) than the central absorbent zone, since the additional comfort zones are more likely to contact the baby's skin. These different features thus reduce undesired friction with the baby's skin while still providing holes for breathability. Thus, in response to this consumer feedback the improved zonal topsheet herein may disclose additional comfort zones around a perimeter of the central absorbent zone that are visually discernable from the central absorbent zone. The additional comfort zones may have these desired characteristics or features (e.g. softer, smaller apertures, smoother, etc.).

Additionally, it was recognized that since various features (e.g. leg cuffs, waistband, solid excrement pocket, etc.) may be positioned on the topsheet, the design of the zonal topsheet may need to take these various features into account. Specifically, it was determined that the various zones of the zonal topsheet may be placed along the topsheet with these other features in mind, so to ensure that these features do not obscure the visually discernable zones of the zonal topsheet. Such obscuring of the visually discernable zones of the improved zonal topsheet may undermine one of the underlying goals of the zonal topsheet, namely to provide multiple topsheet zones which are visually discernable to the consumer. Additionally, since the visually discernable zones provide different functionality (e.g. different degree of smoothness, smaller apertures, etc.), the obscuring of these zones would also deprive the benefit of the unique functionality (e.g. increased smoothness for those zones in increased contact with the skin) that is provided by each zone.

In a first set of embodiments, an absorbent article is provided. The absorbent article includes a first end edge and a second end edge, where the absorbent article has a longitudinal length, L, between the first end edge and the second end edge. The absorbent article also includes a first side edge, a second side edge, a central lateral axis extending in a transverse direction from the first side edge to the second side edge and a central longitudinal axis extending in a longitudinal direction from the first end edge to the second end edge. The absorbent article also includes a first leg cuff comprising a first longitudinal inboard edge facing toward the central longitudinal axis and a second leg cuff comprising a second longitudinal inboard edge facing toward the central longitudinal axis. A width between the first longitudinal inboard edge and the second longitudinal inboard edge, measured in a direction parallel to the central lateral axis, is W. The absorbent article also includes a liquid permeable topsheet having a wearer facing surface and a garment facing surface, a liquid impermeable backsheet, and an absorbent core disposed at least partially between the topsheet and the backsheet. The topsheet comprises at least two visually discernible zones comprising a first zone and a second zone. The first zone has a first zone length that is 0.5 L to L, where the first zone has a minimum width along the first zone length that is in the range of 0.7 W to 1.3 W. The first zone has a different three-dimensionality than the second zone, according to the void-volume planar test. The first zone may also have a different softness than the second zone. In one example, the three-dimensionality (e.g. based on the void-volume planar test) is used as an indicator of a level of smoothness (e.g. low three-dimensionality corresponds to high smoothness, and high three-dimensionality corresponds to low smoothness, etc.). The first zone may also have a different fluid handling properties than the second zone. The first and second zones each comprise apertures and/or three-dimensional features on the wearer facing surface and/or the garment facing surface. The three-dimensional features comprise morphological features (e.g., tufts, projections, embossments) that are at least partially concave or convex.

These and other features, aspects, and advantages of various embodiments will become better understood with reference to the following description, figures, and claims.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a plan view of an example absorbent article in the form of a taped diaper, garment-facing surface facing the viewer, in a flat laid-out state;

FIG. 2 is a plan view of the example absorbent article of FIG. 1, wearer-facing surface facing the viewer, in a flat laid-out state;

FIG. 3 is a front perspective view of the absorbent article of FIGS. 1 and 2 in a fastened position;

FIG. 4 is a front perspective view of an absorbent article in the form of a pant;

FIG. 5 is a rear perspective view of the absorbent article of FIG. 4;

FIG. 6 is a plan view of the absorbent article of FIG. 4, laid flat, with a garment-facing surface facing the viewer;

FIG. 7 is a cross-sectional view of the absorbent article taken about line 7-7 of FIG. 6;

FIG. 8 is a cross-sectional view of the absorbent article taken about line 8-8 of FIG. 6;

FIG. 9 is a plan view of an example absorbent core or an absorbent article;

FIG. 10 is a cross-sectional view, taken about line 10-10, of the absorbent core of FIG. 9;

FIG. 11 is a cross-sectional view, taken about line 11-11, of the absorbent core of FIG. 9;

FIG. 12 is a plan view of an example absorbent article of the present disclosure that is a sanitary napkin;

FIG. 13 is a plan view of an example absorbent article in the form of a taped diaper, garment-facing surface facing the viewer, in a flat laid-out state;

FIG. 14 is a plan view of an example zonal topsheet of an absorbent article;

FIGS. 15a through 15c are plan views of example zonal topsheets of the absorbent article of FIG. 1;

FIGS. 16a and 16b are plan views of example zonal topsheets overlaid by leg cuffs and a solid excrement pocket of the absorbent article of FIG. 1;

FIGS. 17a through 17k are plan views of example zonal topsheets of the absorbent article of FIG. 1;

FIGS. 18a and 18b are side views of an example a void volume of three dimensional features formed in the zonal topsheet that is measured by the planar test;

FIGS. 18c and 18d are side views of an example a void volume of a tuft and projection, respectively, formed in the zonal topsheet and measured by the planar test;

FIGS. 18e and 18f are side views of an example of a tuft and projection, respectively, formed in the zonal topsheet;

FIGS. 19a through 19d are plan views of an example of spacing between apertures formed in the zonal topsheet;

FIGS. 20a through 20c are plan views of an example of parameter values of apertures formed in the zonal topsheet;

FIG. 21 is a photograph illustrating a nonwoven produced according to Example 1;

FIG. 22 is a photograph illustrating a nonwoven produced according to Example 2;

FIG. 23 is a photograph illustrating a nonwoven produced according to Example 3;

FIG. 24 is a photograph illustrating a nonwoven produced according to Example 4;

FIG. 25 is a photograph illustrating a nonwoven produced according to Example 5;

FIG. 26 is a photograph illustrating a nonwoven produced according to Example 6;

FIG. 27 is a photograph illustrating a nonwoven produced according to Example 7;

FIG. 28 is a photograph illustrating a nonwoven produced according to Example 8;

FIG. 29 is a photograph illustrating a nonwoven produced according to Example 9; and

FIG. 30 is a photograph illustrating a nonwoven produced according to Example 10.

### DETAILED DESCRIPTION

Various non-limiting forms of the present disclosure will now be described to provide an overall understanding of the principles of the structure, function, manufacture, and use of the zonal topsheet for an absorbent article disclosed herein. One or more examples of these non-limiting forms are illustrated in the accompanying drawings. Those of ordinary skill in the art will understand that the zonal topsheet for an absorbent article described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### General Description of an Absorbent Article

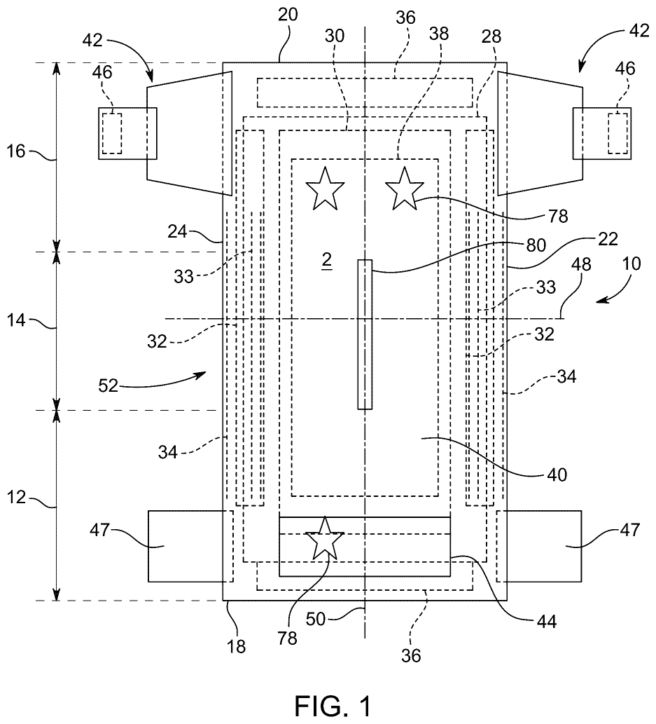

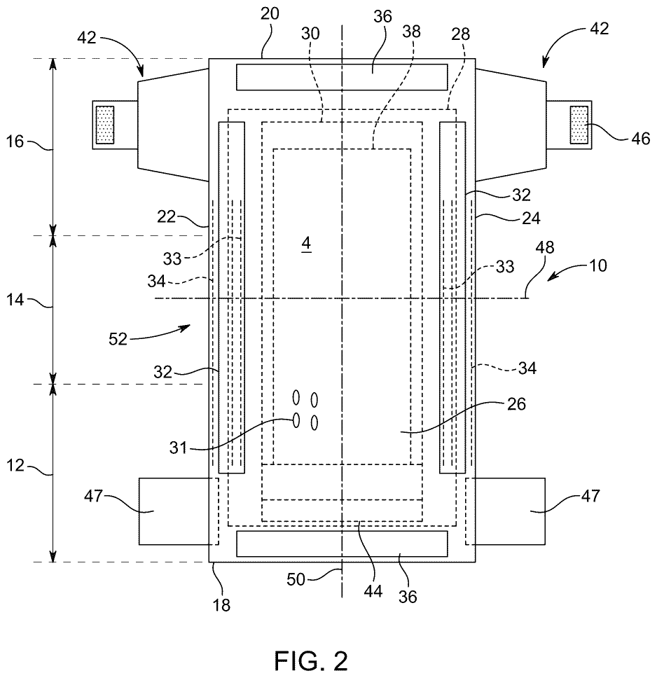

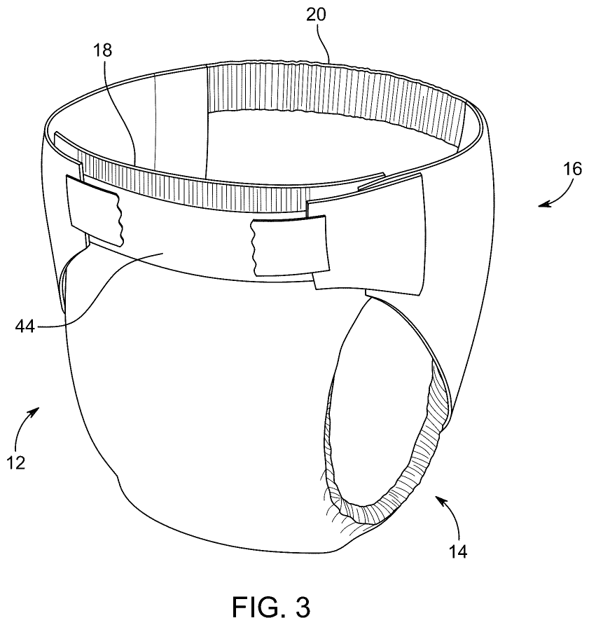

An example absorbent article 10 according to the present disclosure, shown in the form of a taped diaper, is represented in FIGS. 1-3. FIG. 1 is a plan view of the example absorbent article 10, garment-facing surface 2 facing the viewer in a flat, laid-out state (i.e., no elastic contraction). FIG. 2 is a plan view of the example absorbent article 10 of FIG. 1, wearer-facing surface 4 facing the viewer in a flat, laid-out state. FIG. 3 is a front perspective view of the absorbent article 10 of FIGS. 1 and 2 in a fastened configuration. The absorbent article 10 of FIGS. 1-3 is shown for illustration purposes only as the present disclosure may be used for making a wide variety of diapers, including adult incontinence products, pants, or other absorbent articles, such as sanitary napkins and absorbent pads, for example.

The absorbent article 10 may comprise a front waist region 12, a crotch region 14, and a back waist region 16. The crotch region 14 may extend intermediate the front waist region 12 and the back waist region 16. The front wait region 12, the crotch region 14, and the back waist region 16 may each be ⅓ of the length of the absorbent article 10. The absorbent article 10 may comprise a front end edge 18, a back end edge 20 opposite to the front end edge 18, and longitudinally extending, transversely opposed side edges 22 and 24 defined by the chassis 52.

The absorbent article 10 may comprise a liquid permeable topsheet 26, a liquid impermeable backsheet 28, and an absorbent core 30 positioned at least partially intermediate the topsheet 26 and the backsheet 28. The absorbent article 10 may also comprise one or more pairs of barrier leg cuffs 32 with or without elastics 33, one or more pairs of leg elastics 34, one or more elastic waistbands 36, and/or one or more acquisition materials 38. The acquisition material or materials 38 may be positioned intermediate the topsheet 26 and the absorbent core 30. An outer cover material 40, such as a nonwoven material, may cover a garment-facing side of the backsheet 28. The absorbent article 10 may comprise back ears 42 in the back waist region 16. The back ears 42 may comprise fasteners 46 and may extend from the back waist region 16 of the absorbent article 10 and attach (using the fasteners 46) to the landing zone area or landing zone material 44 on a garment-facing portion of the front waist region 12 of the absorbent article 10. The absorbent article 10 may also have front ears 47 in the front waist region 12. The absorbent article 10 may have a central lateral (or transverse) axis 48 and a central longitudinal axis 50. The central lateral axis 48 extends perpendicular to the central longitudinal axis 50.

In other instances, the absorbent article may be in the form of a pant having permanent or refastenable side seams. Suitable refastenable seams are disclosed in U.S. Pat. Appl. Pub. No. 2014/0005020 and U.S. Pat. No. 9,421,137.

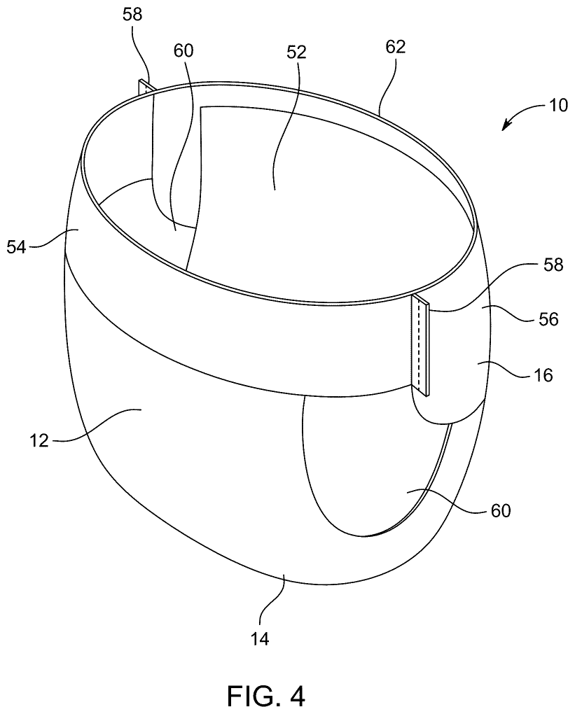

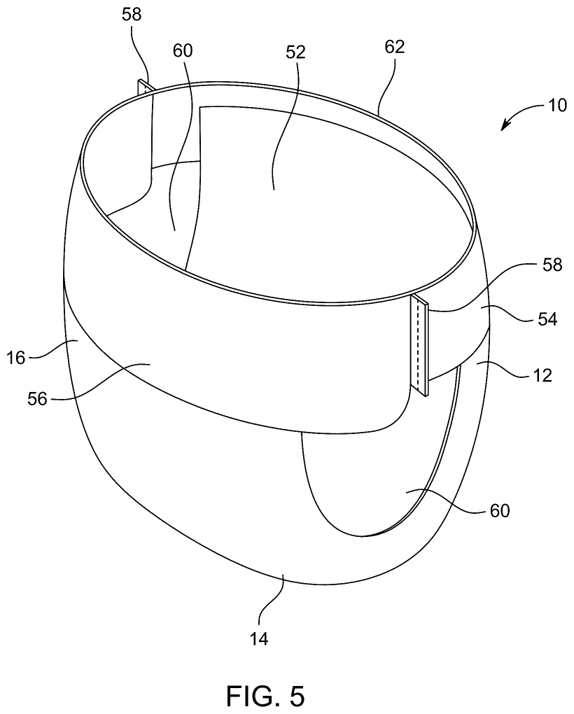

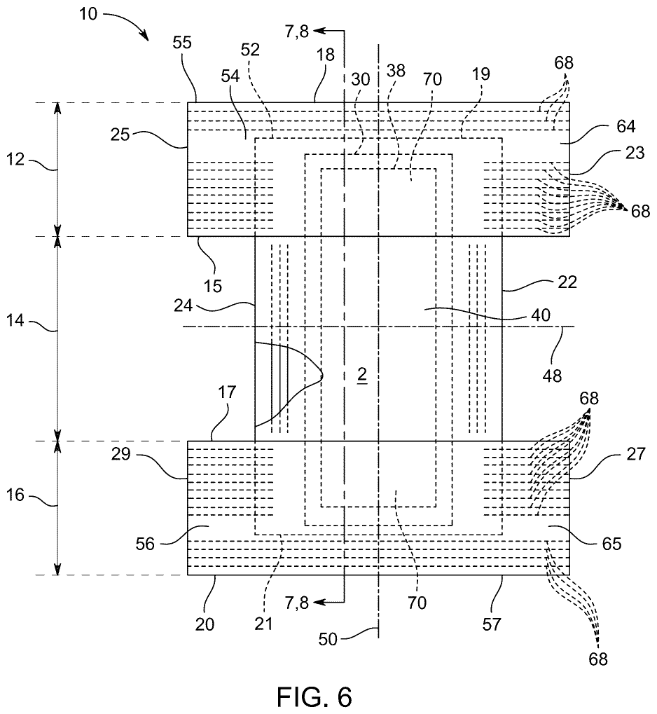

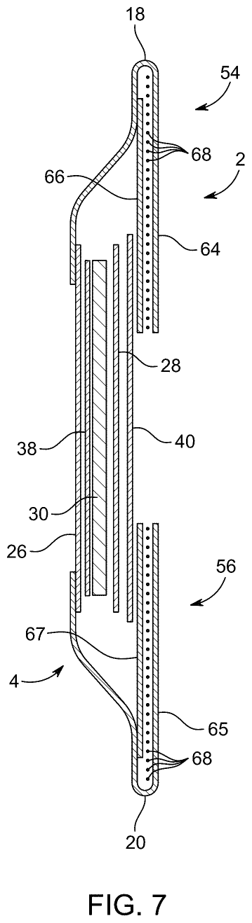

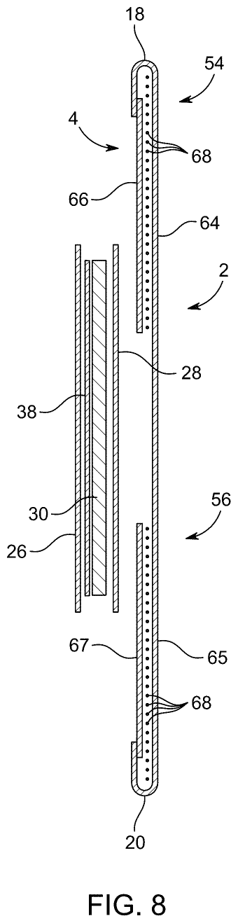

Referring to FIGS. 4-8, an example absorbent article 10 in the form of a pant is illustrated. FIG. 4 is a front perspective view of the absorbent article 10. FIG. 5 is a rear perspective view of the absorbent article 10. FIG. 6 is a plan view of the absorbent article 10, laid flat, with the garment-facing surface facing the viewer. Elements of FIG. 4-8 having the same reference number as described above with respect to FIGS. 1-3 may be the same element (e.g., absorbent core 30). FIG. 7 is an example cross-sectional view of the absorbent article taken about line 7-7 of FIG. 6. FIG. 8 is an example cross-sectional view of the absorbent article taken about line 8-8 of FIG. 6. FIGS. 7 and 8 illustrate example forms of front and back belts 54, 56. The absorbent article 10 may have a front waist region 12, a crotch region 14, and a back waist region 16. Each of the regions 12, 14, and 16 may be ⅓ of the length of the absorbent article 10. The absorbent article 10 may have a chassis 52 (sometimes referred to as a central chassis or central panel) comprising a topsheet 26, a backsheet 28, and an absorbent core 30 disposed at least partially intermediate the topsheet 26 and the backsheet 28, and an optional acquisition material 38, similar to that as described above with respect to FIGS. 1-3. The absorbent article 10 may comprise a front belt 54 in the front waist region 12 and a back belt 56 in the back waist region 16. The chassis 52 may be joined to a wearer-facing surface 4 of the front and back belts 54, 56 or to a garment-facing surface 2 of the belts 54, 56. Side edges 23 and 25 of the front belt 54 may be joined to side edges 27 and 29, respectively, of the back belt 56 to form two side seams 58. The side seams 58 may be any suitable seams known to those of skill in the art, such as butt seams or overlap seams, for example. When the side seams 58 are permanently formed or refastenably closed, the absorbent article 10 in the form of a pant has two leg openings 60 and a waist opening circumference 62. The side seams 58 may be permanently joined using adhesives or bonds, for example, or may be refastenably closed using hook and loop fasteners, for example.

### Belts

Referring to FIGS. 7 and 8, the front and back belts 54 and 56 may comprise front and back inner belt layers 66 and 67 and front and back outer belt layers 64 and 65 having an elastomeric material (e.g., strands 68 or a film (which may be apertured)) disposed at least partially therebetween. The elastic elements 68 or the film may be relaxed (including being cut) to reduce elastic strain over the absorbent core 30 or, may alternatively, run continuously across the absorbent core 30. The elastics elements 68 may have uniform or variable spacing therebetween in any portion of the belts. The elastic elements 68 may also be pre-strained the same amount or different amounts. The front and/or back belts 54 and 56 may have one or more elastic element free zones 70 where the chassis 52 overlaps the belts 54, 56. In other instances, at least some of the elastic elements 68 may extend continuously across the chassis 52.

The front and back inner belt layers 66, 67 and the front and back outer belt layers 64, 65 may be joined using adhesives, heat bonds, pressure bonds or thermoplastic bonds. Various suitable belt layer configurations can be found in U.S. Pat. No. 9,072,632.

Front and back belt end edges 55 and 57 may extend longitudinally beyond the front and back chassis end edges 19 and 21 (as shown in FIG. 6) or they may be co-terminus. The front and back belt side edges 23, 25, 27, and 29 may extend laterally beyond the chassis side edges 22 and 24. The front and back belts 54 and 56 may be continuous (i.e., having at least one layer that is continuous) from belt side edge to belt side edge (e.g., the transverse distances from 23 to 25 and from 27 to 29). Alternatively, the front and back belts 54 and 56 may be discontinuous from belt side edge to belt side edge (e.g., the transverse distances from 23 to 25 and 27 to 29), such that they are discrete.

As disclosed in U.S. Pat. No. 7,901,393, the longitudinal length (along the central longitudinal axis 50) of the back belt 56 may be greater than the longitudinal length of the front belt 54, and this may be particularly useful for increased buttocks coverage when the back belt 56 has a greater longitudinal length versus the front belt 54 adjacent to or immediately adjacent to the side seams 58.

The front outer belt layer 64 and the back outer belt layer 65 may be separated from each other, such that the layers are discrete or, alternatively, these layers may be continuous, such that a layer runs continuously from the front belt end edge 55 to the back belt end edge 57. This may also be true for the front and back inner belt layers 66 and 67—that is, they may also be longitudinally discrete or continuous. Further, the front and back outer belt layers 64 and 65 may be longitudinally continuous while the front and back inner belt layers 66 and 67 are longitudinally discrete, such that a gap is formed between them—a gap between the front and back inner and outer belt layers 64, 65, 66, and 67 is shown in FIG. 7 and a gap between the front and back inner belt layers 66 and 67 is shown in FIG. 8. In some configurations shown in FIGS. 7 and 8, portions of the outer belt layers 64, 65 may be folded over onto the inner belt layers, respectively. In addition, as shown in FIG. 7, portions of the outer belt layers 64, 65 may also be folded over onto the chassis 52.

The front and back belts 54 and 56 may include slits, holes, and/or perforations providing increased breathability, softness, and a garment-like texture. Underwear-like appearance can be enhanced by substantially aligning the waist and leg edges at the side seams 58 (see FIGS. 4 and 5).

The front and back belts 54 and 56 may comprise graphics (see e.g., 78 of FIG. 1). The graphics may extend substantially around the entire circumference of the absorbent article 10 and may be disposed across side seams 58 and/or across proximal front and back belt edges 15 and 17; or, alternatively, adjacent to the seams 58 and/or proximal front and back belt edges 15 and 17 in the manner described in U.S. Pat. No. 9,498,389 to create a more underwear-like article. The graphics may also be discontinuous.

Alternatively, instead of attaching belts 54 and 56 to the chassis 52 to form a pant, discrete side panels may be attached to side edges of the chassis 22 and 24. Suitable forms of pants comprising discrete side panels are disclosed in U.S. Pat. Nos. 6,645,190; 8,747,379; 8,372,052; 8,361,048; 6,761,711; 6,817,994; 8,007,485; 7,862,550; 6,969,377; 7,497,851; 6,849,067; 6,893,426; 6,953,452; 6,840,928; 8,579,876; 7,682,349; 7,156,833; and 7,201,744.

### Topsheet

The topsheet 26 is the part of the absorbent article 10 that is in contact with the wearer's skin. The topsheet 26 may be joined to portions of the backsheet 28, the absorbent core 30, the barrier leg cuffs 32, and/or any other layers as is known to those of ordinary skill in the art. The topsheet 26 may be compliant, soft-feeling, and non-irritating to the wearer's skin. Further, at least a portion of, or all of, the topsheet may be liquid permeable, permitting liquid bodily exudates to readily penetrate through its thickness. A suitable topsheet may be manufactured from a wide range of materials, such as porous foams, reticulated foams, apertured plastic films, woven materials, nonwoven materials, woven or nonwoven materials of natural fibers (e.g., wood or cotton fibers), synthetic fibers or filaments (e.g., polyester or polypropylene or bicomponent PE/PP fibers or mixtures thereof), or a combination of natural and synthetic fibers. The topsheet may have one or more layers. The topsheet may be apertured (FIG. 2, element 31), may have any suitable three-dimensional features, and/or may have a plurality of embossments (e.g., a bond pattern). The topsheet may comprise a variable basis weight nonwoven material, such as those described in U.S. Pat. Appl. No. 2017/0191198. The topsheet may be apertured by overbonding a material and then rupturing the overbonds through ring rolling, such as disclosed in U.S. Pat. No. 5,628,097, to Benson et al., issued on May 13, 1997, and disclosed in U.S. Pat. Appl. Publication No. US 2016/0136014 to Arora et al. Any portion of the topsheet may be coated with a skin care composition, an antibacterial agent, a surfactant, and/or other beneficial agents. The topsheet may be hydrophilic or hydrophobic or may have hydrophilic and/or hydrophobic portions or layers. If the topsheet is hydrophobic, typically apertures will be present so that bodily exudates may pass through the topsheet. The topsheet may comprise a one or more layer hydroentangled material with or without apertures. The topsheet may comprise a variable basis weight nonwoven material.

### Backsheet

The backsheet 28 is generally that portion of the absorbent article 10 positioned proximate to the garment-facing surface of the absorbent core 30. The backsheet 28 may be joined to portions of the topsheet 26, the outer cover material 40, the absorbent core 30, and/or any other layers of the absorbent article by any attachment methods known to those of skill in the art. The backsheet 28 prevents, or at least inhibits, the bodily exudates absorbed and contained in the absorbent core 10 from soiling articles such as bedsheets, undergarments, and/or clothing. The backsheet is typically liquid impermeable, or at least substantially liquid impermeable. The backsheet may, for example, be or comprise a thin plastic film, such as a thermoplastic film having a thickness of about 0.012 mm to about 0.051 mm. Other suitable backsheet materials may include breathable materials, such as films, which permit vapors to escape from the absorbent article, while still preventing, or at least inhibiting, bodily exudates from passing through the backsheet. The backsheet 28 may be coterminous with the outer cover material 40.

### Outer Cover Material

The outer cover material (sometimes referred to as a backsheet nonwoven) 40 may comprise one or more nonwoven materials joined to the backsheet 28 and that covers the backsheet 28. The outer cover material 40 forms at least a portion of the garment-facing surface 2 of the absorbent article 10 and effectively “covers” the backsheet 28 so that film is not present on the garment-facing surface 2. The outer cover material 40 may comprise a bond pattern, apertures, and/or three-dimensional features. The outer cover material 40 may be a hydroentangled nonwoven material or a variable basis weight nonwoven material. Such a material may have one or more layers.

### Absorbent Core

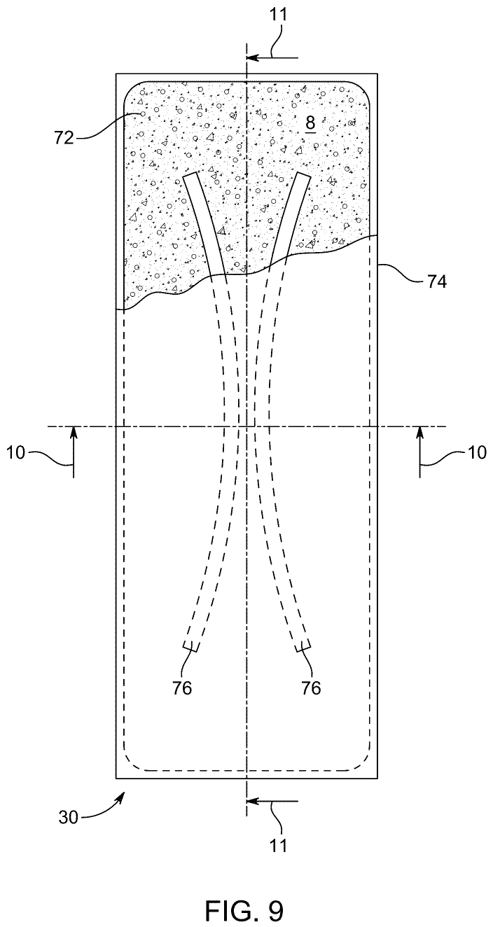

As used herein, the term “absorbent core” refers to a component of the absorbent article 10 for absorbing and containing liquid such as urine received by the absorbent article. The absorbent core thus typically has a high absorbent capacity. An example absorbent core 30 is schematically shown in FIGS. 9-11. The absorbent core comprises an absorbent material 72. The absorbent typically comprises a core wrap 74 that encloses or sandwiches the absorbent material.

The core wrap may be a single material that is folded and attached to itself, or it may comprise a separate top layer and bottom layer that may be bonded, adhesively joined, or otherwise joined together. The top and bottom layers of the core wrap may be the same or different. The absorbent material typically comprises superabsorbent particles which are optionally mixed with cellulose fibers. As used herein, “absorbent core” does not include any acquisition-distribution systems, topsheet, or backsheet of the absorbent article.

The example absorbent core 30 shown in isolation in FIGS. 9-11 is in the dry state (before use). The absorbent core may typically have a generally rectangular shape as defined by its longitudinal edges and transversal front edge and back edge or may have other shapes.

Absorbent material 72 may be deposited as an absorbent layer having a generally rectangular outline, as represented in FIG. 9. A wide variety of absorbent cores may also be used. The absorbent material 72 layer may also have a non-rectangular perimeter (“shaped” core), in particular, the absorbent material 72 may define a tapering along its width towards the central region of the core (or “dog-bone” shape). In this way, the absorbent material deposition area may have a relatively narrow width in an area of the core intended to be placed in the crotch region of the absorbent article. This may provide for example better wearing comfort. Other shapes can also be used such as a “T” or “Y” or “hourglass” for the area of the absorbent material.

The absorbent material 72 may be any conventional absorbent material known in the art. For example, the absorbent material may comprise a blend of cellulose fibers and superabsorbent particles (“SAP”), typically with the percentage of SAP ranging from about 50% to about 75% by weight of the absorbent material. The absorbent material may also be free of cellulose fibers, as is known in so-called airfelt-free cores, where the absorbent material consists, or consists essentially, of SAP. The absorbent material may also be a high internal phase emulsion foam. High loft nonwoven materials may be present in the core bags, or proximate but outside the core bags. The absorbent material may comprise one or more layers of a coform material. The coform material may comprise a mixture of fibers and an absorbent material. The fibers may comprise staple fibers such as synthetic fibers or absorbent fibers. The synthetic fibers may comprise polypropylene fibers, polyethylene fibers, and/or bicomponent fibers. The absorbent fibers may comprise pulp, lyocell, and/or viscose. The fibers may also comprise thermoplastic filaments (scattered or interconnected networks). The absorbent material may comprise a super absorbent polymeric material in fiber or particle form. The one or more layers of coform material may comprise virgin or recycled materials. The staple fibers may be present in the coform material in an amount of from about 5 wt. % to about 50 wt. %. The absorbent material may be present in the nonwoven web in an amount of from about 50 wt. % to about 95 wt. %. The staple fibers may have an average length of from about 5 mm to about 50 mm. The staple fibers and absorbent fibers may be thermally bonded or hydraulically entangled to form the nonwoven web (as disclosed in WO2023/022979).

“Superabsorbent polymer” or “SAP” refers herein to absorbent materials, typically cross-linked polymeric materials, that can absorb at least 10 times their weight of an aqueous 0.9% saline solution as measured using the Centrifuge Retention Capacity (CRC) test (EDANA method WSP 241.2.R3 (12)). The SAP may in particular have a CRC value of at least 20 g/g, in particular of from about 20 g/g to about 40 g/g. “Superabsorbent polymer particles”, as used herein, refers to a superabsorbent polymer material which is in particulate form so as to be flowable in the dry state.

Various absorbent core designs comprising high amounts of SAP have been proposed in the past, see for example in U.S. Pat. No. 5,599,335 (Goldman), EP1,447,066 (Busam), WO95/11652 (Tanzer), U.S. Pat. Appl. Pub. No. 2008/0312622A1 (Hundorf), WO2012/052172 (Van Malderen). In particular, the SAP printing technology as disclosed in U.S. Pat. No. 7,838,722 (Blessing), U.S. Pat. Nos. 9,072,634 and 8,206,533 (both to Hundorf et al.) may be used. The present disclosure however is not limited to a particular type of absorbent core. The absorbent core may also comprise one or more glues such as an auxiliary glue applied between the internal surface of one (or both) of the core wrap layers and the absorbent material to reduce leakage of SAP outside the core wrap. A micro-fibrous adhesive net may also be used in air-felt free cores as described in the above Hundorf references. These glues are not represented in the Figures for simplicity. Other core constructions comprising a high loft nonwoven substrate, such as a carded nonwoven layer, having a porous structure into which SAP particles have been deposited, may also be used in present disclosure.

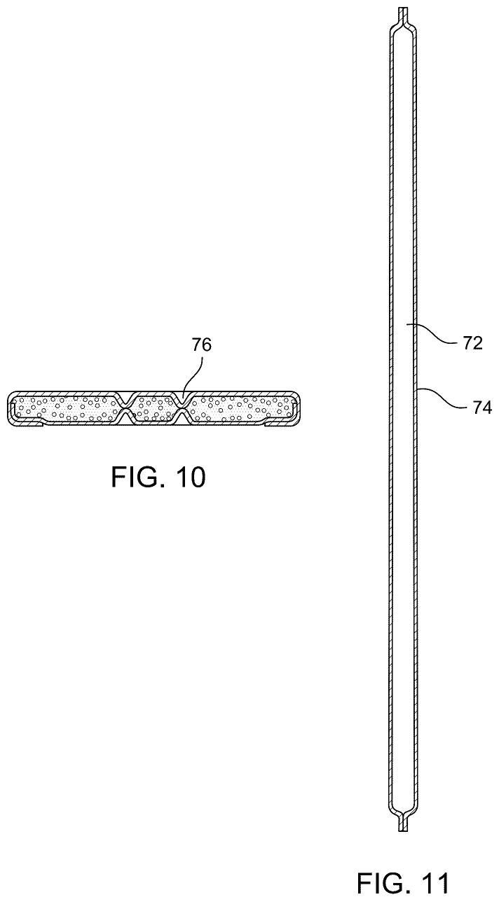

The absorbent material may be deposited as a continuous layer within the core wrap. The absorbent material may also be present discontinuously, for example, as individual pockets or stripes of absorbent material enclosed within the core wrap and separated from each other by material-free junction areas. A continuous layer of absorbent material, in particular of SAP, may also be obtained by combining two absorbent layers having matching discontinuous absorbent material application pattern, wherein the resulting layer is substantially continuously distributed across the absorbent particulate polymer material area, as illustrated in FIGS. 10-11. As for example taught in U.S. Pat. Appl. Pub. No. 2008/0312622A1 (Hundorf), each absorbent material layer may thus comprise a pattern having absorbent material land areas and absorbent material-free junction areas, wherein the absorbent material land areas of the first layer correspond substantially to the absorbent material-free junction areas of the second layer and vice versa.

The basis weight (amount deposited per unit of surface) of the absorbent material may also be varied to create a profiled distribution of absorbent material, in particular in the longitudinal direction to provide more absorbency towards the center and the middle of the core, but also in the transversal direction, or both directions of the core. The absorbent core may also comprise one or more longitudinally (or otherwise) extending channels 76, which are areas of the absorbent layer substantially free of absorbent material within the absorbent material layer. The top side of the core wrap may be advantageously bonded to the bottom side of the core by adhesive, mechanical or ultra-sonic bonding through these material-free areas. Example disclosures of such channels in an airfelt-free core can be found in U.S. Pat. No. 9,789,011. One or more channels may also be formed in absorbent cores comprising a mix of cellulose fibers and SAP particles. These channels may embody any suitable shapes and any suitable number of channels may be provided. In other instances, the absorbent core may be embossed to create the impression of channels, with absorbent material remaining within the channels. The absorbent core in FIGS. 9-11 is merely an example absorbent core. Many other absorbent cores with or without channels are also within the scope of the present disclosure.

### Barrier Leg Cuffs/Leg Elastics

Referring to FIGS. 1 and 2, for example, the absorbent article 10 may comprise one or more pairs of barrier leg cuffs 32 and one or more pairs of leg elastics 34. The barrier leg cuffs 32 may be positioned laterally inboard of leg elastics 34. Each barrier leg cuff 32 may be formed by a piece of material which is bonded to the absorbent article 10 so it can extend upwards from a wearer-facing surface 4 of the absorbent article 10 and provide improved containment of body exudates approximately at the junction of the torso and legs of the wearer. The barrier leg cuffs 32 are delimited by a proximal edge joined directly or indirectly to the topsheet and/or the backsheet and a free terminal edge, which is intended to contact and form a seal with the wearer's skin. The barrier leg cuffs 32 may extend at least partially between the front end edge 18 and the back end edge 20 of the absorbent article 10 on opposite sides of the central longitudinal axis 50 and may be at least present in the crotch region 14. The barrier leg cuffs 32 may each comprise one or more elastics 33 (e.g., elastic strands or strips) near or at the free terminal edge. These elastics 33 cause the barrier leg cuffs 32 to help form a seal around the legs and torso of a wearer. The leg elastics 34 extend at least partially between the front end edge 18 and the back end edge 20. The leg elastics 34 essentially cause portions of the absorbent article 10 proximate to the chassis side edges 22, 24 to help form a seal around the legs of the wearer. The leg elastics 34 may extend at least within the crotch region 14. The barrier leg cuffs 32 may comprise one or more layers of nonwoven material. The nonwoven material may be hydroentangled.

### Elastic Waistband

Referring to FIGS. 1 and 2, the absorbent article 10 may comprise one or more elastic waistbands 36. The elastic waistbands 36 may be positioned on the garment-facing surface 2 or the wearer-facing surface 4. Alternatively, the elastic waistbands may be positioned intermediate the topsheet and the backsheet. As an example, a first elastic waistband 36 may be present in the front waist region 12 near the front end edge 18 and a second elastic waistband 36 may be present in the back waist region 16 near the back end edge 20. The elastic waistbands 36 may aid in sealing the absorbent article 10 around a waist of a wearer and at least inhibiting bodily exudates from escaping the absorbent article 10 through the waist opening circumference. In some instances, an elastic waistband may fully surround the waist opening circumference of an absorbent article. The elastic waistband may comprise an elastic film joined to the topsheet and a nonwoven material covering the elastic film. In other instances, the elastic waistband may comprise an elastic film sandwiched between two nonwoven materials. The elastic film may be ultrasonically bonded, or otherwise bonded or attached, to the one or more nonwoven materials. The one or more nonwoven materials may be hydroentangled. The elastic film and/or the nonwoven materials may be preactivated (i.e., activated prior to being joined together) or the formed elastic film/nonwoven laminate may be activated post laminate formation.

### Acquisition Materials

Referring to FIGS. 1, 2, 7, and 8, one or more acquisition materials 38 may be present at least partially intermediate the topsheet 26 and the absorbent core 30. The acquisition materials 38 are typically hydrophilic materials that provide significant wicking of bodily exudates. These materials may dewater the topsheet 26 and quickly move bodily exudates into the absorbent core 30. The acquisition materials 38 may comprise one or more nonwoven materials, foams, formed films, apertured formed films, cellulosic materials, cross-linked cellulosic materials, air laid cellulosic nonwoven materials, spunlace materials, or combinations thereof, for example. The acquisition material may be rectangular or may be shaped, such as hourglass shaped. Typically, an acquisition material 38 may have a width and length that are smaller than the width and length of the topsheet 26. The acquisition material may be a secondary topsheet in the feminine pad context. The acquisition materials may have one or more channels as described above with reference to the absorbent core 30 (including the embossed version). The channels in the acquisition material may align or not align with channels in the absorbent core 30. In an example, a first acquisition material may comprise a nonwoven material and as second acquisition material may comprise a cross-linked cellulosic material.

In one example, the acquisition materials 38 may be combined with the topsheet 26 to form some of the three-dimensional features, such as the morphological features (e.g. tuft 232, projection 234, etc.) discussed herein.

### Landing Zone

Referring to FIGS. 1 and 2, the absorbent article 10 may have a landing zone area 44 that is formed in a portion of the garment-facing surface 2 of the outer cover material 40. The landing zone area 44 may be in the back waist region 16 if the absorbent article 10 fastens from front to back or may be in the front waist region 12 if the absorbent article 10 fastens back to front. In some instances, the landing zone 44 may be or may comprise one or more discrete nonwoven materials that are attached to a portion of the outer cover material 40 in the front waist region 12 or the back waist region 16 depending upon whether the absorbent article fastens in the front or the back. In essence, the landing zone 44 is configured to receive the fasteners 46 and may comprise, for example, a plurality of loops configured to be engaged with, a plurality of hooks on the fasteners 46, or vice versa.

### Wetness Indicator/Graphics

Referring to FIG. 1, the absorbent articles 10 of the present disclosure may comprise graphics 78 and/or wetness indicators 80 that are visible from the garment-facing surface 2. The graphics 78 may be printed on the landing zone 40, the backsheet 28, and/or at other locations. The wetness indicators 80 are typically applied to the absorbent core facing side of the backsheet 28, so that they can be contacted by bodily exudates within the absorbent core 30. In some instances, the wetness indicators 80 may form portions of the graphics 78. For example, a wetness indicator may appear or disappear and create/remove a character within some graphics. In other instances, the wetness indicators 80 may coordinate (e.g., same design, same pattern, same color) or not coordinate with the graphics 78.

### Front and Back Ears

Referring to FIGS. 1 and 2, as referenced above, the absorbent article 10 may have front and/or back ears 47, 42 in a taped diaper context. Only one set of ears may be required in most taped diapers. The single set of ears may comprise fasteners 46 configured to engage the landing zone or landing zone area 44. If two sets of ears are provided, in most instances, only one set of the ears may have fasteners 46, with the other set being free of fasteners. Any ears with fasteners may be configured to engage an opposing ear or the landing zone or landing zone area 44. One set of ears may comprise primary fasteners and the other set of ears may comprise secondary fasteners. The ears, or portions thereof, may be elastic or may have elastic panels. In an example, an elastic film or elastic strands may be positioned intermediate a first nonwoven material and a second nonwoven material. One or more of the first and second nonwoven materials may be hydroentangled. The elastic film may or may not be apertured. The ears may be shaped. The ears may be integral (e.g., extension of the outer cover material 40, the backsheet 28, and/or the topsheet 26) or may be discrete components attached to a chassis 52 of the absorbent article on a wearer-facing surface 4, on the garment-facing surface 2, or intermediate the two surfaces 4, 2. The back ears may comprise an elastic film sandwiched between two nonwoven materials forming a laminate. The elastic film and/or the nonwoven materials may be pre-activated (i.e., activated prior to being joined together) and the laminate may be joined by ultrasonic bonds. Such laminates are disclosed in U.S. Pat. No. 10,485,713. In other instances, the elastic film may not be preactivated and instead the laminate may be activated after the laminate is formed. Such laminates may also be ultrasonically bonded.

### Masking Layer

One or more masking layers or materials may be provided in the absorbent articles 10. A masking layer may be a layer that provides a cushiony feel when the absorbent article is touched from the garment-facing surface 2 or the wearer-facing surface 4. The masking layer may “mask” a grainy feel potentially caused by the absorbent material 72, such as superabsorbent polymers. The masking layer may “mask” bodily exudates from being visible when viewing the wearer-facing surface 4 or the garment-facing surface 2 of the absorbent article 10. The masking layer may have a basis weight in the range of about 15 gsm to about 50 gsm or about 15 gsm to about 40 gsm. The masking layer may comprise one or more nonwoven materials (e.g., a hydroentangled nonwoven material), foams, pulp layers, and/or other suitable materials. The masking layer may be the outer cover material 40. The masking layer may be the layer forming the garment-facing side or the wearer-facing side of the core bag 74. The masking layer may be a separate material positioned intermediate the garment-facing side of the core bag 74 and the liquid impermeable backsheet 28.

### Packages

The absorbent articles of the present disclosure may be placed into packages. The packages may comprise polymeric films, paper, and/or other materials. Graphics and/or indicia relating to properties of the absorbent articles may be formed on, printed on, positioned on, and/or placed on outer portions of the packages. Each package may comprise a plurality of absorbent articles. The absorbent articles may be packed under compression so as to reduce the size of the packages, while still providing an adequate amount of absorbent articles per package. By packaging the absorbent articles under compression, caregivers can easily handle and store the packages, while also providing distribution savings to manufacturers owing to the size of the packages. The packages may comprise polymeric films comprising recycled material, such as about 20% to about 100%, about 30% to about 90%, about 30% to about 80%, about 40% to about 60%, or about 50% recycled material. The recycled material may comprise post-industrial recycled material (PIR) and/or post-consumer recycled material (PCR). In some instances, the polymeric films used for the packages may comprise two outer layers and one or more inner layers. The one or more inner layers may comprise the recycled material or may comprise more recycled material than the outer layers. The recycled material may comprise recycled polyethylene. The recycled material may comprise recycled polyethylene PIR from trim from the packaging operation.

The package material may comprise paper, paper based material, paper with one or more barrier layers, or a paper/film laminate. The package material may be in the range of about 50 gsm to about 100 gsm or about 70 gsm to about 90 gsm and the one or more barrier layers may be in the range of about 3 gsm to about 15 gsm. The paper based package material with or without one or more barrier layers may exhibit a machine direction tensile strength of at least 5.0 kN/m, a machine direction stretch of at least 3 percent, a cross-machine direction tensile strength of at least 3 kN/m, and a cross-direction stretch at break of at least 4 percent, each as determined via ISO 1924-3.

The paper based package material or paper based package material comprising a barrier layer or film may be recyclable or recyclable in normal paper recycling operations. The recyclability extent of the paper based package may be determined via recyclable percentage. The paper based package of the present disclosure may exhibit recyclable percentages of 70 percent or greater, 80 percent or greater, or 90 percent or greater. The paper based package of the present disclosure may have a recyclable percentage of between 70 percent to about 99.9 percent, between about 80 percent to about 99.9 percent, or between about 90 percent to about 99.9 percent. In one example, the package material of the present disclosure may exhibit a recyclable percentage of from about 95 percent to about 99.9 percent, from about 97 percent to about 99.9 percent, or from about 98 percent to about 99.9 percent. The recyclable percentage of the paper based package may be determined via test PTS-RH: 021/97 (Draft October 2019) under category II, as performed by Papiertechnische Stiftung located at Pirnaer Strasse 37, 01809 Heidenau, Germany. In another instance, the paper based packages of the present disclosure may exhibit an overall “pass” test outcome as determined by PTS-RH: 021/97 (Draft October 2019) under category II method. Any of the paper based packages may have opening features, such as lines of perforation, and may also have handles.

### Arrays

“Array” means a display of packages comprising disposable absorbent articles of different article constructions (e.g., different elastomeric materials [compositionally and/or structurally] in the side panels, back ears, side flaps and/or belts flaps, different graphic elements, different product structures, fasteners, waistbands, or lack thereof). The packages may have the same brand and/or sub-brand and/or the same trademark registration and/or having been manufactured by or for a common manufacturer and the packages may be available at a common point of sale (e.g. oriented in proximity to each other in a given area of a retail store). An array is marketed as a line-up of products normally having like packaging elements (e.g., packaging material type, film, paper, dominant color, design theme, etc.) that convey to consumers that the different individual packages are part of a larger line-up. Arrays often have the same brand, for example, “Huggies,” and same sub-brand, for example, “Pull-Ups.” A different product in the array may have the same brand “Huggies” and the sub-brand “Little Movers.” The differences between the “Pull-Ups” product of the array and the “Little Movers” product in the array may include product form, application style, different fastening designs or other structural elements intended to address the differences in physiological or psychological development. Furthermore, the packaging is distinctly different in that “Pull-Ups” is packaged in a predominately blue or pink film bag and “Little Movers” is packaged in a predominately red film bag.

Further regarding “Arrays,” as another example an array may be formed by different products having different product forms manufactured by the same manufacturer, for example, “Kimberly-Clark”, and bearing a common trademark registration for example, one product may have the brand name “Huggies,” and sub-brand, for example, “Pull-Ups.” A different product in the array may have a brand/sub-brand “Good Nites” and both are registered trademarks of The Kimberly-Clark Corporation and/or are manufactured by Kimberly-Clark. Arrays also often have the same trademarks, including trademarks of the brand, sub-brand, and/or features and/or benefits across the line-up. “On-line Array” means an “Array” distributed by a common on-line source.

### Sanitary Napkin

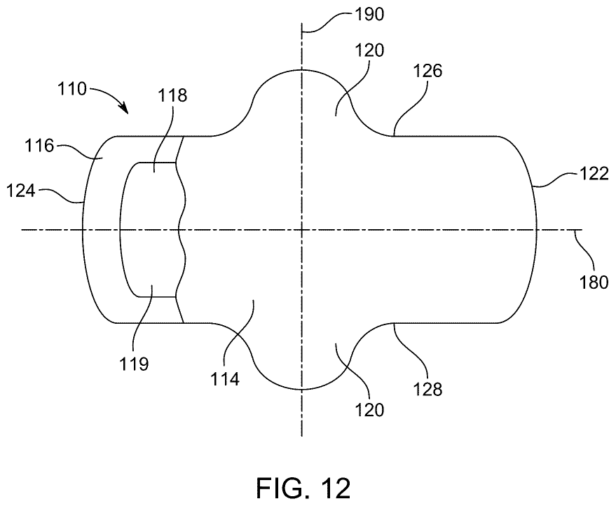

Referring to FIG. 12, an absorbent article of the present disclosure may be a sanitary napkin 110. The sanitary napkin 110 may comprise a liquid permeable topsheet 114, a liquid impermeable, or substantially liquid impermeable, backsheet 116, and an absorbent core 118. The liquid impermeable backsheet 116 may or may not be vapor permeable. The absorbent core 118 may have any or all of the features described herein with respect to the absorbent core 30 and, in some forms, may have a secondary topsheet 119 (STS) instead of the acquisition materials disclosed above. The STS 119 may comprise one or more channels, as described above (including the embossed version). In some forms, channels in the STS 119 may be aligned with channels in the absorbent core 118. The sanitary napkin 110 may also comprise wings 120 extending outwardly with respect to a longitudinal axis 180 of the sanitary napkin 110. The sanitary napkin 110 may also comprise a lateral axis 190. The wings 120 may be joined to the topsheet 114, the backsheet 116, and/or the absorbent core 118. The sanitary napkin 110 may also comprise a front edge 122, a back edge 124 longitudinally opposing the front edge 122, a first side edge 126, and a second side edge 128 longitudinally opposing the first side edge 126. The longitudinal axis 180 may extend from a midpoint of the front edge 122 to a midpoint of the back edge 124. The lateral axis 190 may extend from a midpoint of the first side edge 128 to a midpoint of the second side edge 128. The sanitary napkin 110 may also be provided with additional features commonly found in sanitary napkins as is known in the art.

### Fasteners

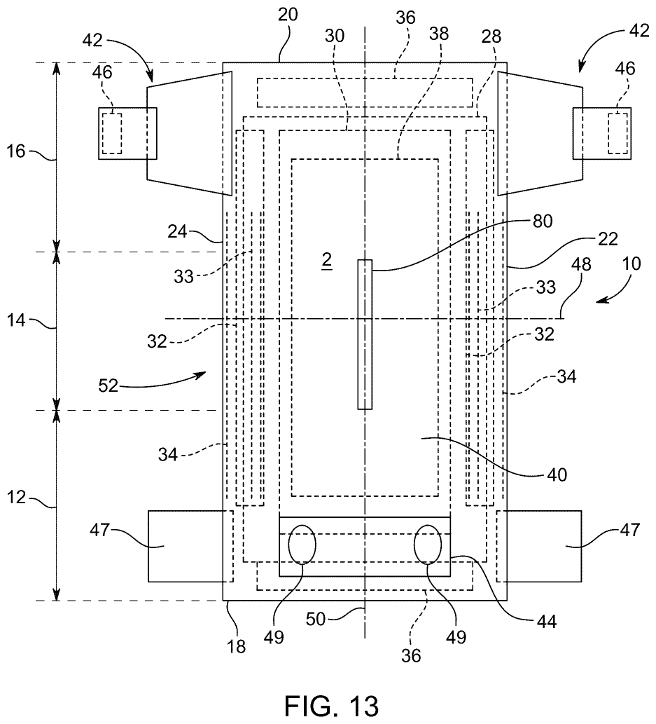

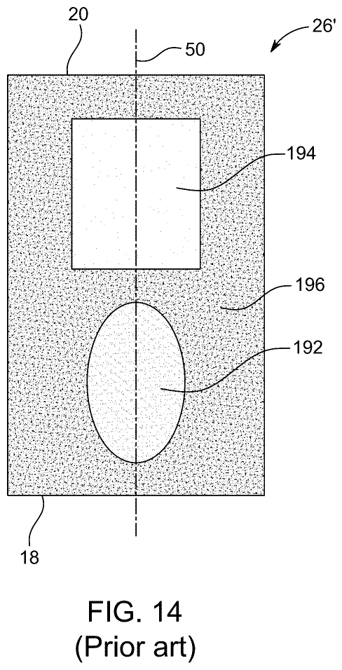

Referring to FIG. 13, the back ears 42 may comprise fasteners 46. The fasteners 46 may be configured to cooperate with the landing zone area or landing zone material 44 on the garment-facing surface of the front waist region 12 of the absorbent article 10. Additionally, the absorbent article may comprise one or more secondary fasteners 49. The secondary fasteners 49 may be disposed on the outer cover material 40 or a component of the absorbent article, such as the landing zone 44 as illustrated in FIG. 13. The secondary fasteners may be a separate material joined to the absorbent article. The secondary fasteners may be integrally formed from a material of the absorbent article. For example, the secondary fasteners may comprise one or more hooks or protrusions formed from the material of the outer cover material 40, the landing zone material 44, or a film. The hooks or protrusions may be formed using the process described in U.S. Pat. No. 8,784,722 to Rocha et al. The secondary fasteners may be configured to cooperate with a portion of the back ears. For example, a secondary fastener comprising hooks or protrusions are configured to engage the nonwoven material of the back ear. It is to be appreciated that the secondary fasteners may be any mechanical fastener that is configured to cooperate with a component of the absorbent article.

### Bio-Based Content for Components

Components of the absorbent articles described herein may at least partially be comprised of bio-based content as described in U.S. Pat. Appl. No. 2007/0219521A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and U.S. Pat. Nos. 10,166,312 and 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. No. 11,433,158, issued on Sep. 6, 2022.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

### Improved Zonal Topsheet

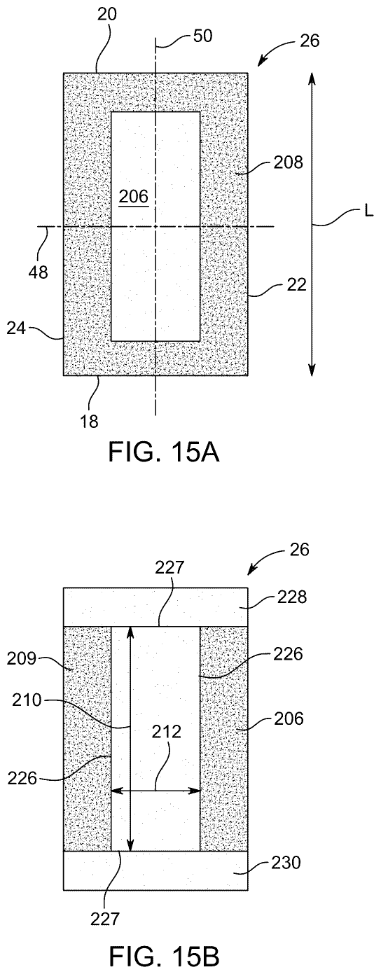

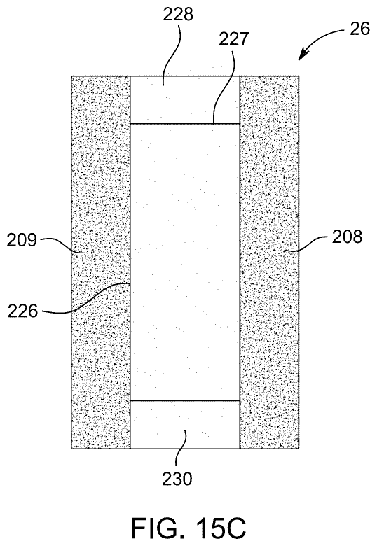

An improved zonal topsheet for use with an absorbent article will now be discussed. FIGS. 15a through 15c are plan views of example zonal topsheets 26 of the absorbent article 10 of FIG. 1. The absorbent article 10 may be a diaper (e.g., a taped diaper or a pant diaper for babies or adults).

The liquid permeable topsheet 26 of FIGS. 15a through 15c may have the wearer facing surface 4 and the garment facing surface 2. As shown in FIGS. 15a through 15c, the topsheet 26 may have at least two visually discernible zones. For purposes of this description, “visually discernable” means that the multiple zones of the topsheet can be visually discerned by the consumer of the absorbent article, due to one or more of a color difference between the zones, a difference in a pattern between the zones, a difference in a texture (e.g. three-dimensionality of 3D features) between the zones and/or a difference in tactile properties, etc. These two or more visually discernable zones may include a first zone 206 and a second zone 208 encompassing a perimeter of the first zone 206, as shown in FIG. 15a. The first zone 206 and the second zone 208 may differ in that each zone has different three-dimensionality (e.g. different three dimensional structures) and/or different permeability (e.g. different aperture characteristics such as size, density, etc.) and/or different tactile properties and/or different color. In another example, the first zone 206 and the second zone 208 may have a different three-dimensionality such as a different core height value and/or a different core void volume, according to the Surface Topography Test disclosed herein.

In one example, the first zone 206 may be provided along the central longitudinal axis 50 and is intended for absorption (e.g. of liquid excrement and solid excrement). The second zone 208 may be provided along a perimeter of at least one side of the first zone 206. As shown in FIG. 15a, in one example the second zone 208 may surround/encompass the first zone 206. In one example, the second zone 208 may extend to one or more of the front end edge 18, rear end edge 20, first side edge 22 and/or second side edge 24 of the zonal topsheet 26. However, in other examples, the second zone 208 does not extend to one or more of the front end edge 18, rear end edge 20, first side edge 22 and/or second side edge 24. For purposes of this description, the first zone 206 may be referred to as an “absorbent zone” and the other zones of the zonal topsheet 26 (e.g. zones 208, 209, 228, 230) may be referred to as “comfort zones” since the first zone 206 is provided primarily to absorb waste material whereas the other zones 208, 209, 228, 230 are provided primarily to provide comfort to the wearer (due to their increased contact with the skin as compared to the first zone 206).

In an example, the first zone 206 may be made from material that may be very soft, yet very resistant/resilient (long lasting). In another example, the first zone 206 may have a pattern that may be textured with apertures. In one example, the apertures in the first zone 206 are denser (or bigger) than in the surrounding zones discussed herein. In one example, the apertures formed in the first zone 206 may be larger than formed apertures in the other zones. Additionally, in another example, holes or apertures formed in the first zone 206 are denser (or bigger) than apertures formed in zones of the topsheet 26 surrounding the first zone 206. In yet another example, the material of the first zone 206 of the zonal topsheet 26 may be thicker and/or more padded than the material of the other zones of the zonal topsheet 26 that surround the first zone 206.

Since the second zone 208 is more proximate to the regions of the absorbent article (e.g. front end edge 18, rear end edge 20, first side edge 22, second side edge 24) engaging the skin, the second zone 208 may have increased contact with the skin than the first zone 206. Thus, it is desired that the second zone 208 have different characteristics than the first zone 206 (e.g. different three dimensionality, different aperture characteristics and thus different permeability, different smoothness, etc.). In one example, the second zone 208 may have characteristics to maximize comfort and/or minimize friction with the skin (e.g. reduced three dimensionality of 3D features in the zone, smaller apertures, etc.) and promote breathability (e.g. apertures). In an example, the first zone 206 may have characteristics to maximize absorption of liquid excrement and solid excrement (e.g. increased three dimensionality, larger apertures, etc.).

In one example, the first zone 206 has an area between 5,000 mm² and 50,000 mm², preferably between 10,000 mm² and 25,000 mm².

Additionally, in another example, the first zone 206 may be formed of a single material. Alternatively, in another example, the topsheet 26 including the first zone 206 may include two materials, where the two materials may be joined by heat, chemistry, and/or mechanical force.

In an example, a lotion may be provided on the topsheet 26. In one example, the first zone 206 has a first amount of lotion and the second zone 208 has a second amount of lotion where the first amount of lotion is different form the second amount of lotion.

In an example, the first zone 206 may have a different pattern than the second zone 208. In some examples, the zones of the zonal topsheet 26 may use subtle and aesthetic features (e.g. flowers, clouds, hearts, channels, etc.) on each zone to visually distinguish each zone of the zonal topsheet 26. In still other examples, the zones of the zonal topsheet 26 may use functional features (e.g. channels) on each zone to visually distinguish each zone of the zonal topsheet 26. These subtle and aesthetic features advantageously may not affect functionality of each zone of the zonal topsheet 26.

In one example, the front waist region 12 and back waist regions 16 of the zonal topsheet 36 are mainly designed for softness and breathability. In another example, the front and back waist regions 12, 16 could also provide leakage barriers. For example, the baby's belly is especially in contact with the wearer-facing surface 4 (inner-layer) in these regions 12, 16 due to a tight fit around the waist. Thus, it is desirable that the material of the zonal topsheet 26 in the front and back waist regions 12, 16 may be extra gentle, avoid frictions and let skin breathe. In another example, the material of the front and back waist regions 12, 16 of the zonal topsheet 26 may be cushiony, smooth, permit “bouncing back” and/or be flexible/stretchable (fit), since lots of skin contact may occur in these regions 12, 16. In yet another example, a pattern of small holes may be provided in the front and back waist regions 12, 16 of the zonal topsheet 26, with no or limited 3D features to maximize skin contact while providing air breathability and minimize skin abrasion/friction. Additionally, in another example the front and back waist regions 12, 16 of the zonal topsheet 36 has a balanced thickness-enough to provide a leakage barrier, yet not too thick to cause discomfort to the wearer.

Although FIG. 15a depicts an example of the topsheet 26 where the second zone 208 may surround the first zone 206, in other examples more than one comfort zone may surround/encompass the perimeter of the first zone 206. As shown in FIG. 15b, the second zone 208 may be provided along one side of the first zone 206 and a third zone 209 may be provided along an opposite side of the first zone 206 from the second zone 208. Thus, in this example, the first zone 206 may be positioned at least partially intermediate the second zone 208 and the third zone 209. In an example, the third zone 209 may be similar to the second zone 208 and/or may have similar characteristics (e.g. three dimensionality, aperture characteristics, etc.) as the second zone 208. In one example, the second zone 208 of FIG. 15b may be provided proximate the first side edge 22 and the third zone 209 may be provided proximate the second side edge 24.

In an example, the second and third zones 208, 209 of the zonal topsheet 26 may provide more comfort and flexibility around the legs of the baby. In one example, this may be achieved by having more gliding softness, a different pattern/material thickness on these zones 208, 209 (e.g. smaller holes for breathability, thinner material for comfort, etc.) than the first zone 206.

As further shown in FIG. 15b, the zonal topsheet 26 may feature a fourth zone 228 provided along a top of the first zone 206 and a fifth zone 230 provided along a bottom of the first zone 206. As with the second and third zones 208, 209, in this example the first zone 206 may be positioned at least partially intermediate the fourth zone 228 and fifth zone 230. In an example, the fourth and fifth zones 228, 230 may have similar characteristics (e.g. three dimensionality, aperture characteristics, etc.) as the second zone 208 since the fourth and fifth zones 228, 230 similarly have close contact with the skin. In one example, the fourth zone 228 of FIG. 15b may be provided proximate the rear end edge 20 of the topsheet 26 and the fifth zone 230 may be provided proximate the front end edge 18 of the topsheet 26.

As previously discussed, the zonal topsheet 26 features one or more visually discernable zones. In one example, one or more visually discernable transition areas may be provided between the central zone 206 and each of the adjacent comfort zones. For example, as shown in FIG. 15b a transition area 226 may be provided between the first and third zones 206, 209 and a similar transition area 226 may be provided between the first and second zones 206, 208. Additionally, as shown in FIG. 15b a transition area 227 may be provided between the first and fourth zones 206, 228 and a similar transition area 227 may be provided between the first and fifth zones 206, 230. In an example, each of these transition areas 226, 227 indicate a transition between visually discernable criteria (e.g. color, texture, visual pattern, aperture characteristics, etc.) such that the consumer can decipher the visually discernable zones across the transition areas 226, 227.

FIG. 15c depicts another example of a topsheet 26 that is similar to the topsheet 26 of FIG. 15b, with the exception that the second and third zones 208, 209 of the topsheet 26 of FIG. 15c extend from the top to the bottom of the topsheet 26 (e.g. from the rear end edge 20 to the front end edge 18 of the absorbent article 10) whereas the second and third zones 208, 209 of the topsheet 26 of FIG. 15b do not extend from the top to the bottom of the topsheet 26. Similarly, the fourth and fifth zones 228, 230 of the topsheet 26 of FIG. 15c do not extend from the first side to the second side of topsheet 26 (e.g. from the first side edge 22 to the second side edge 24) whereas the fourth and fifth zones 228, 230 of the topsheet 26 of FIG. 15b do extend from the first side to the second side of the topsheet 26.

As previously discussed, the absorbent article 10 may feature structures placed on top of the topsheet 26 (e.g., leg cuffs 32, waistbands 36 and solid excrement pockets). It was determined that when such structures are placed on the topsheet 26, the various zones of the zonal topsheet 26 disclosed herein may need to be properly positioned and/or sized such that when these structures are placed on the topsheet 26, they do not obstruct the consumer from seeing the visually discernable zones of the improved zonal topsheet 26. Thus, one or more characteristics of the zones of the improved zonal topsheet 26 will now be discussed, in light of these structures placed on the topsheet 26, so to ensure that these visually discernable zones may remain visible.

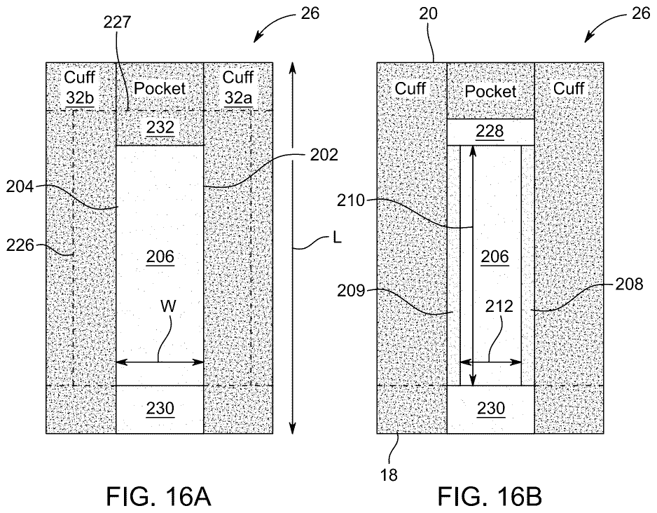

FIGS. 16a and 16b are plan views of example zonal topsheets 26 overlaid by structures (e.g. leg cuffs 32 and a solid excrement pocket 232) of the absorbent article 10 of FIG. 1. In other examples, the structures may include the waistband 36 of the absorbent article 10 that is overlaid on the topsheet 26. As shown in FIGS. 16a and 16b, the absorbent article 10 and topsheet 26 may have a longitudinal length L between the front end edge 18 and the rear end edge 20, measured along the central longitudinal axis 50. Additionally, as shown in FIGS. 16a and 16b, the first leg cuff 32a that may adjoin the first side edge 22 may have a first longitudinal inboard edge 202 and the second leg cuff 32b that may adjoin the second side edge 24 may have a second longitudinal inboard edge 204. A width W that may extend between the first longitudinal inboard edge 202 and the second longitudinal inboard edge 204 is depicted in FIG. 16a and may be measured in a direction parallel to the central lateral axis 48.

In an example, FIG. 16a depicts an undesired positioning and scaling of the zones of the zonal topsheet 26, where the first zone length 210 and first zone width 212 are such that the transition areas 226, 227 of the zonal topsheet 26 are concealed behind the structures (e.g. leg cuffs 32, solid excrement pocket 232, etc.) and thus not discernable to the consumer during use of the absorbent article 10. Thus, it was recognized that the first zone length 210 and first zone width 212 should be each adjusted within a particular range such that the transition areas 226, 227 are discernable by the consumer during use of the absorbent article 10.

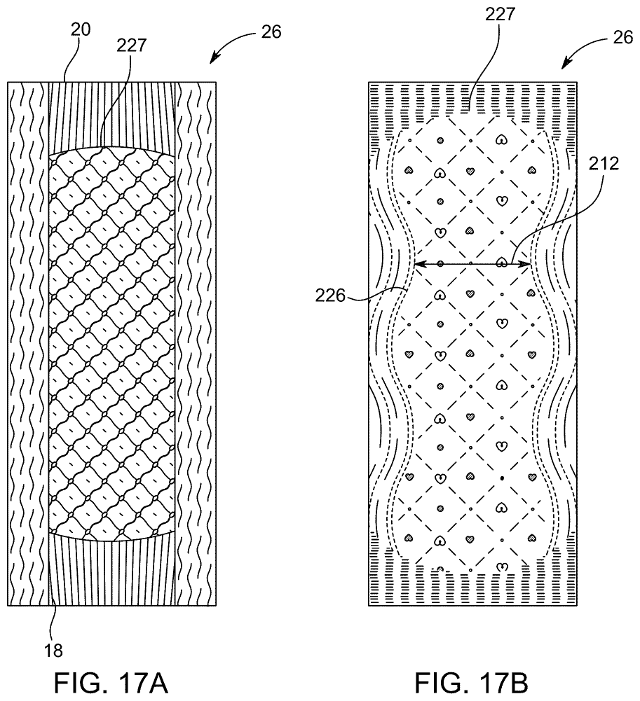

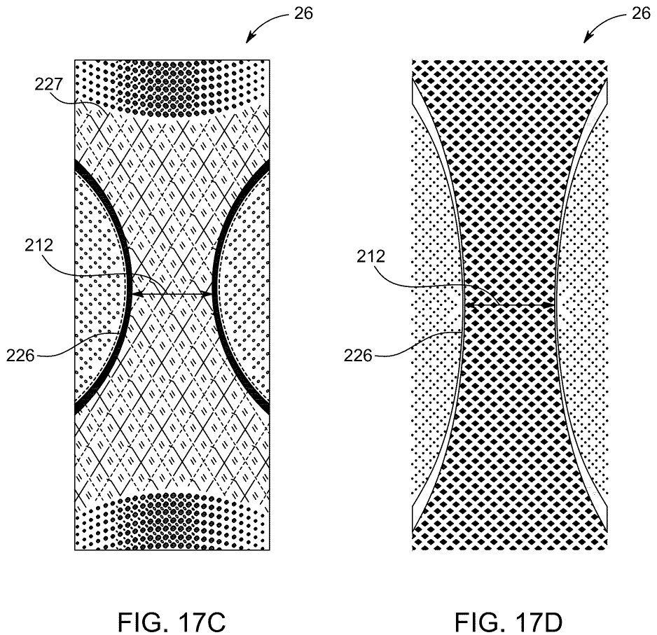

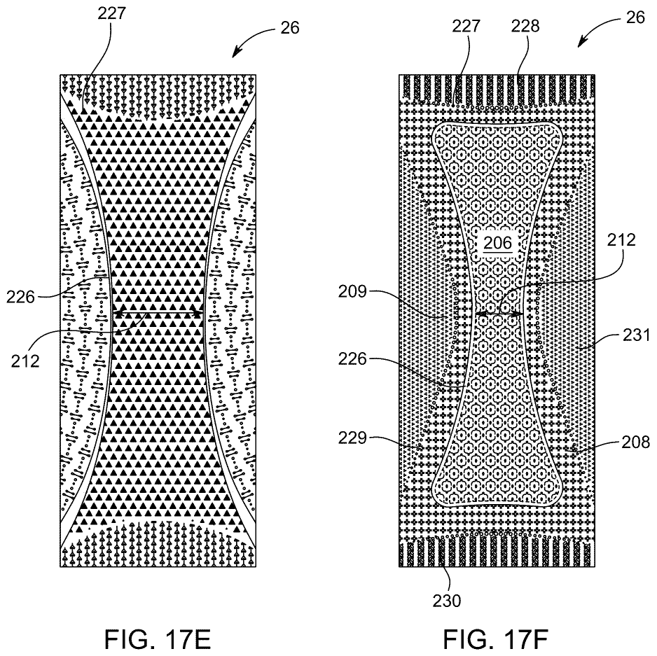

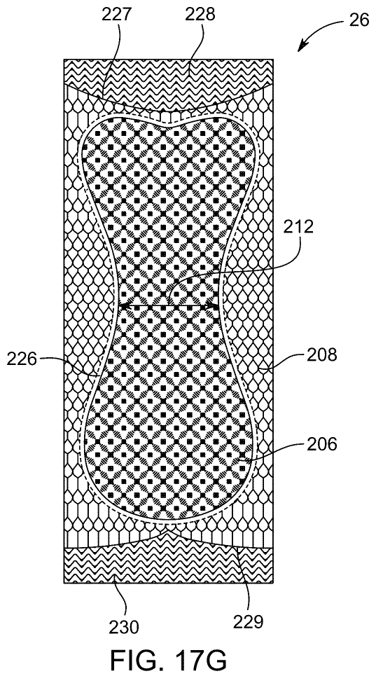

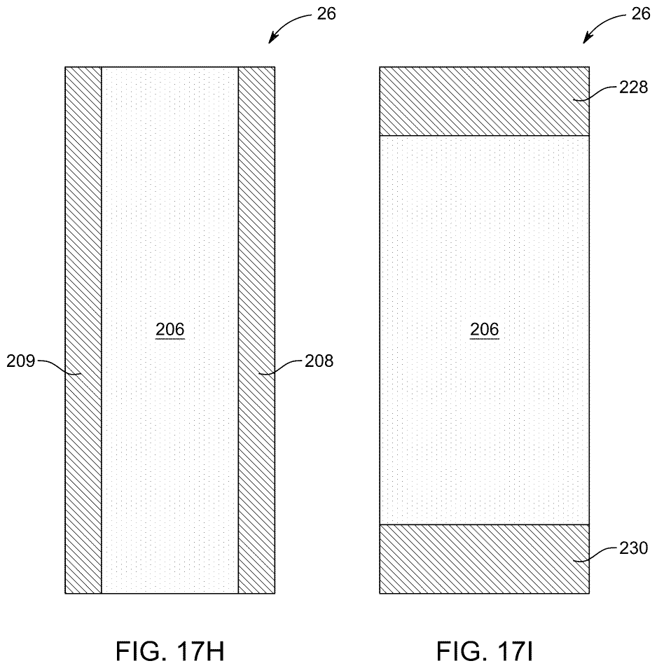

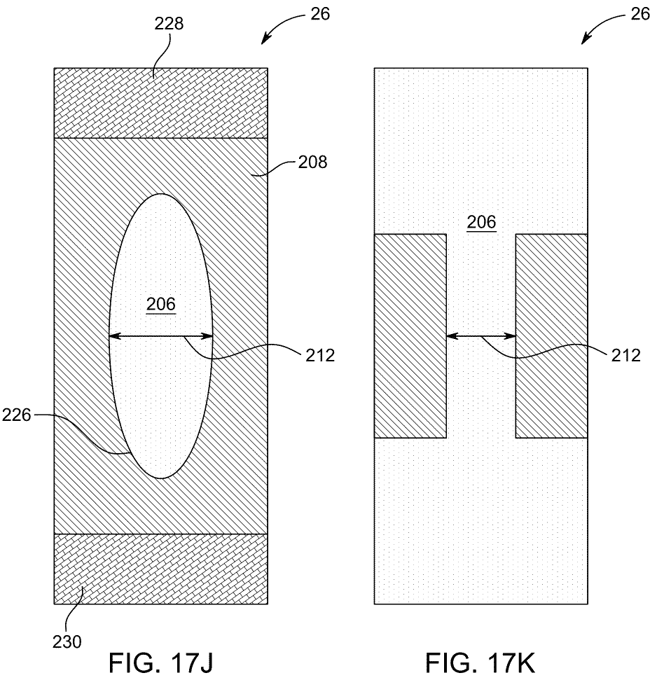

For purposes of this description, “first zone width 212” may mean a minimum value of a width of the first zone 206 along the length 210 of the first zone 206. For the example of FIG. 16b, where the first zone 206 is rectangular in shape, the first zone width 212 is constant along the length 210 of the first zone 206 and thus the first zone width 212 is the value of this constant width. In another example, such as discussed with respect to FIGS. 17a through 17k, where the width of the first zone 206 varies along the first zone length 210, the first zone width 212 is a minimum value of this width along the first zone length 210.

FIG. 16b depicts an example of a desired positioning and scaling of the zones of the zonal topsheet 26. In one example, the first zone 206 may have a first zone length 210 that is in a particular range between a first ratio (e.g. 0.5 L) and a second ratio (e.g. 1 L) of the longitudinal length L. However, in other examples, the first zone length 210 may be outside this range. It was recognized that having the first zone length 210 to be less than this first ratio (e.g. 0.2 L) of the longitudinal length L may be undesirable, as it may not provide a sufficient surface area of absorption. It was also recognized that having the first zone length 210 to be greater than this second ratio (e.g. 1.5 L) of the longitudinal length L may also be undesirable as it may result in the transition areas 227 being concealed behind the solid excrement pocket 232 or a waistband 36 and thus not observable by the consumer during use of the absorbent article 10.

As further shown in FIG. 16b, in one example, the first zone 206 may have a first zone width 212 that is in a particular range between a first ratio (e.g. 0.7 W) and a second ratio (e.g. 1.3 W) of the width W. However, in other examples, the first zone width 212 may be outside this range. It was recognized that having the first zone width 212 to be less than this first ratio (e.g. 0.2 W) of the width W may be undesirable, as it may not provide a sufficient surface area of absorption. It was also recognized that having the first zone width 212 to be greater than this second ratio (e.g. 1.6 W) of the width W may also be undesirable, as it may result in the transition areas 227 being concealed behind the leg cuff 32a, 32b and thus not observable by the consumer during use of the absorbent article 10. In some examples, the first zone width 212 can be slightly greater than the width W (e.g. 1.1 W) and in this desired range, since upon bending the zonal topsheet 26 to place the absorbent article 10 on the baby, the transition area 226 will move inward inside the second longitudinal inboard edge 204 and thus be visually discernable to the consumer.

Although FIGS. 15a through 15c and FIGS. 16a and 16b depict rectangular shaped first zones 206 and thus linear boundary areas 226, 227 between the first zones 206 and the adjacent comfort zones of the zonal topsheet, this is merely one example of a shape which the first zone 206 can take. In other examples, the first zone 206 of the zonal topsheet 26 may feature one or more arcuate surfaces and thus result in at least part of one or more of the transition areas 226, 227 having an arcuate portion. In an example, the curved transition areas 226, 227 are desirable to the consumer as they tend to visually follow the shape of the baby's anatomy (e.g. round shape of the buttocks).

Additionally, in yet another example, the rounded or arcuate transition areas 226, 227 may give the feeling of more coverage for the middle absorbency zone 206. In yet another example, the rounded or curved transition areas 226, 227 may advantageously provide a smooth/seamless transition (e.g. compared to a strong delimitation) which heightens the feeling of maximum coverage. In another example, the transition areas 226, 227 may define channels that provide bending lines for flexibility and/or additional leakage barrier protection.

In one example, FIGS. 17a through 17k are plan views of example zonal topsheets 26 of the absorbent article 10 of FIG. 1, where the first zone 206 features one or more arcuate surfaces which may form one or more of the transition areas 226, 227 with an arcuate portion. For example, FIG. 17a depicts that the first zone 206 may have an arcuate surface adjoining the fourth and fifth zones 228, 230 and thus the transition area 227 therebetween may have an arcuate portion.

In another example, the first zone 206 may be shaped such that the width of the first zone 206 along the length 210 varies. This is depicted in the examples of FIGS. 17b through 17g. As previously discussed, the first zone width 212 of the first zones 206 of FIGS. 17b through 17g may be a minimum value of the width of the first zone 206 along the first zone length 210. In some examples, this minimum first zone width 212 may occur at a midpoint along the first zone length 210 (e.g. FIGS. 17c through 17g). However, in other examples, this minimum first zone width 212 may occur at a portion of the first zone 206 that is offset from the midpoint along the first zone length 210 (e.g. FIG. 17b).

In yet another example, FIGS. 17h through 17k depict other examples of the position, shape and scaling of the various zones of the zonal topsheet 26 that may be employed in forming the absorbent article 10. For the example topsheet 26 of FIG. 17j, which is oval shaped, the first zone width 212 is a maximum value of the width of the first zone 206 along the first zone length 210. In the example topsheet 26 of FIG. 17j, the maximum first zone width 212 occurs at the midpoint along the first zone length 210.

### Three Dimensionality Test

As previously discussed, the different zones of the zonal topsheet 26 may be visually discernable by the transition areas 226, 227 using visual criteria (e.g. color). In some examples, the different zones may also be discernable based on visual and/or tactile features (e.g. different three-dimensionality) which may facilitate functional differences of the zones. In one example, the first zone 206 may have a higher degree of three-dimensionality to provide a certain function of the first zone 206 (e.g. increased absorption) whereas the other zones 208, 209, 228, 230 may have a smaller degree of three-dimensionality to serve a different function (e.g. increased smoothness, comfort, etc) for the other comfort zones that have increased skin contact. In order to assess the three-dimensionality of each zone, a three-dimensional test may be provided. One example of such a three-dimensional test is now discussed.

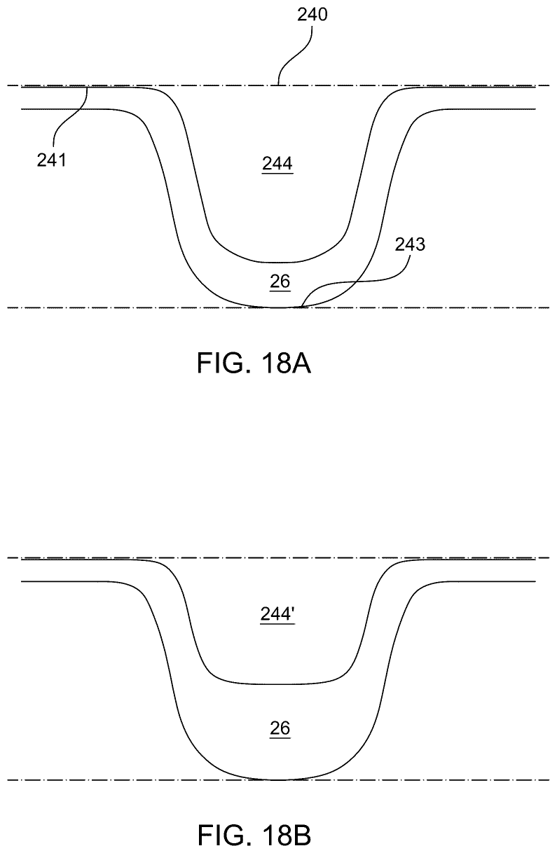

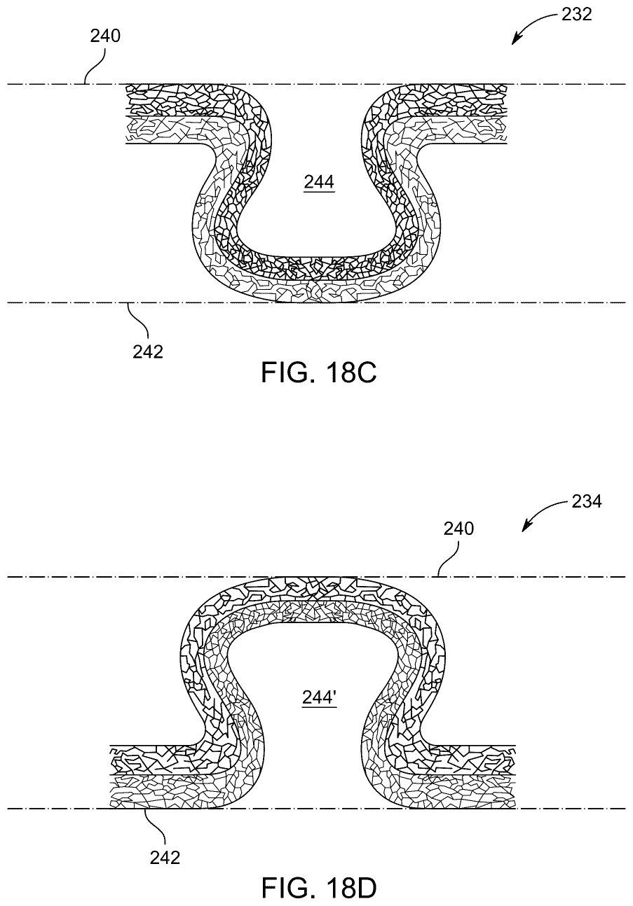

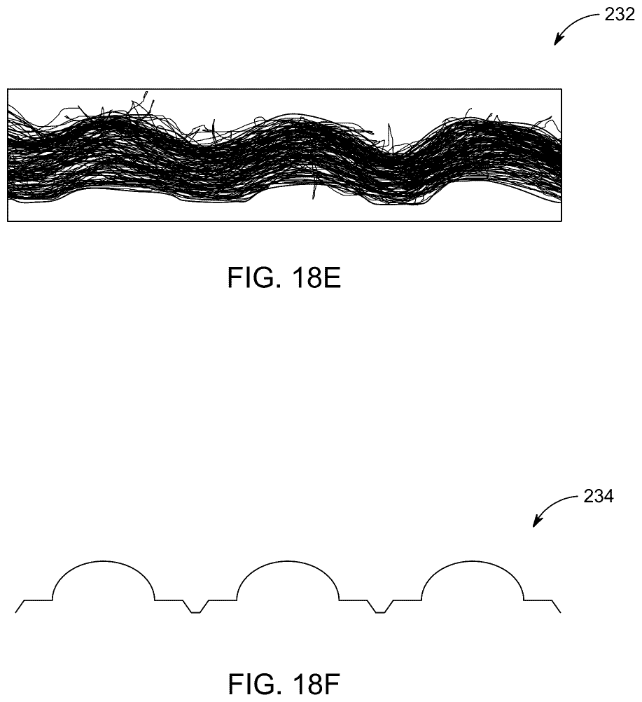

FIGS. 18a and 18b are side views of an example of a void volume 244 of three dimensional features formed in the zonal topsheet 26 that is measured by a planar test. As shown in FIG. 18a, a first plane 240 is provided that intersects a peak surface 241 of the topsheet 26 and a second plane 242 is provided that intersects a trough surface 243 of the topsheet 26. The void volume 244 may be a hollow area of the zonal topsheet 26 between the two planes 240, 242. In one example, a value of the void volume 244 may be used as indicative of the degree of three-dimensionality of the topsheet 26. As further shown in FIG. 18b, the void volume 244′ has decreased from the void volume 244 of FIG. 18a which may be attributed to the zonal topsheet 26 having a smaller degree of three-dimensionality (e.g. shallower valley than FIG. 18a). In some examples, the value of the void volume 244, 244′ of the planar test is expressed in units of volume. In other examples, the value of the void volume 244, 244′ of the planar test is expressed in a percentage value, which indicates a percentage of the volume between the planes 240, 242 that is occupied by the void volume 244, 244′. In some examples, the value of the void volume of the planar test is taken over a fixed or predetermined length of the first zone 206, so to ensure a fair comparison of the void volume between different first zones 206.

In one example, the first zone 206 of the zonal topsheet 26 may have a different three-dimensionality than one or more of the other comfort zones (e.g. zones 208, 209, 228, 230). In one example, the value of the void volume 244 in the first zone 206 may be different than the value of the void volume 244 for one or more of the other comfort zones of the zonal topsheet 26 (e.g. using the above discussed planar test). In some examples, the first zone 206 may have a higher three-dimensionality (e.g. higher value of the void volume 244) than one or more of the other zones of the zonal topsheet 26. In an example, each zone of the zonal topsheet 26 may have some degree of three-dimensionality (e.g. a non-zero value of the void volume 244) and thus in this example none of the zones of the zonal topsheet 26 are entirely flat (e.g. with a zero value of the void volume 244).

In some examples, the smoothness of each zone of the zonal topsheet 26 may be characterized and/or quantified using the three-dimensionality test disclosed herein. Thus, a lower value of the void volume 244 may be used to quantify or characterize a higher degree of smoothness and a higher value of the void volume 244 may be used to quantify or characterize a lower degree of smoothness of the particular zone.

### Three Dimensional Characteristics of Zonal Topsheet

In some examples, one or more of the zones of the zonal topsheet may have three-dimensional features, such as morphological features that are at least partially concave or convex (e.g. tufts, projections, embossments, etc.). One or more of these example morphological features will now be discussed herein.

FIG. 18c is a side view of an example of a tuft 232 three-dimensional feature that may be formed in one or more of the zones of the zonal topsheet 26. Although FIG. 18c depicts the topsheet 26 and tuft 232 with two layers, in other examples the topsheet 26 and tuft 232 may have one layer or more than two layers. In one example, the void volume 244 of the tuft 232 is also depicted, as determined by the planar test of FIGS. 18a and 18b between the first and second planes 240, 242. In yet another example, FIG. 18e depicts an example of tufts 232 formed along the surface of one or more zones of the zonal topsheet 26.

In an example, where tufts 232 are provided in the first zone 206 and in the other comfort zones 208, 209, 228, 230 of the zonal topsheet 26, the value of the void volume 244 of the tufts 232 in the first zone 206 may be greater than the value of the void volume 244 of the tufts 232 in the other comfort zones of the zonal topsheet 26.

FIG. 18d is a side view of an example of a projection 234 three-dimensional feature that may be formed in one or more of the zones of the zonal topsheet 26. Although FIG. 18d depicts the topsheet 26 and projection 234 with two layers, in other examples the topsheet 26 and projection 234 may have one layer or more than two layers. In one example, the void volume 244′ of the projection 234 is also depicted, as determined by the planar test of FIGS. 18a and 18b between the first and second planes 240, 242. FIG. 18f depicts an example of projections 234 formed along the surface of one or more zones of the zonal topsheet 26.

In an example, where projections 234 are provided in the first zone 206 and in the other zones 208, 209, 228, 230 of the zonal topsheet 26, the value of the void volume 244′ of the projections 234 in the first zone 206 may be greater than the value of the void volume 244′ of the projections 234 in the other zones of the zonal topsheet 26.

In some examples, three-dimensional features that go downward beneath the surface of the topsheet 26 (e.g. tufts 232) are preferred to three-dimensional features that extend above the surface of the topsheet 26 (e.g. projections 234) for various functions of the absorbent article 10 (e.g. urine and bowel movement handling). Similarly, in some examples the three-dimensional features (e.g. tufts 232 or projections 234) are preferred to a flat surface for these various functions of the absorbent article 10. In one example, three-dimensional features (e.g. projections 234) that extend up above the topsheet 26 surface may cause frictions with the skin, irritation, and/or imprints.

### Aperture Characteristics of Zonal Topsheet

As previously discussed, the different zones of the zonal topsheet 26 may be visually discernable by the transition areas 226, 227 using visual criteria (e.g. color). In some examples, the different zones may also be discernable based on visual and/or tactile features (e.g. apertures with different characteristics) which may be facilitate functional differences of the zones. In one example, the first zone 206 may have apertures having larger dimensions and/or density to provide a certain function of the first zone 206 (e.g. increased absorption) whereas the other zones 208, 209, 228, 230 may have apertures with smaller dimensions and/or density to serve a different function (e.g. increased smoothness, comfort, etc) for the other zones. One or more aperture characteristics are now discussed, which may differ between the first zone 206 and the other zones of the zonal topsheet 26. In one example, as a result of the difference of the aperture characteristics between the first zone 206 and the other zones, the first zone 206 may have a different permeability than the other zones of the zonal topsheet 26.

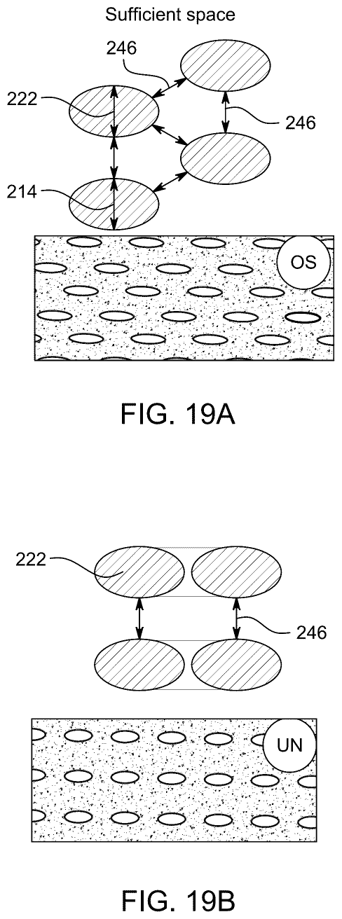

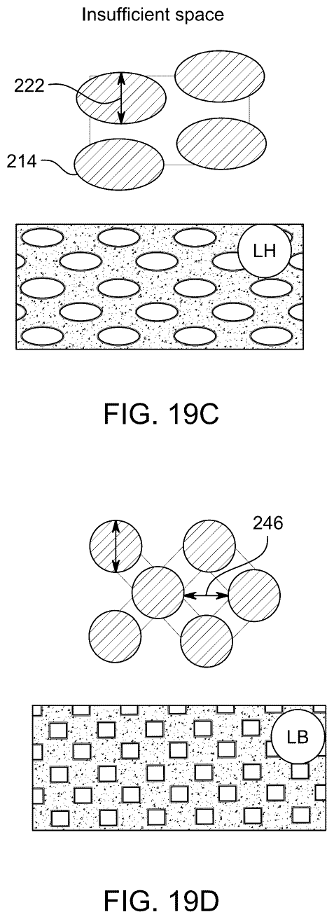

FIGS. 19a through 19d are plant views of an example of spacing 246 between apertures 214 formed in the zonal topsheet 26. In one example, the apertures 214 depicted in FIGS. 19a through 19d are formed in the first zone 206 of the zonal topsheet 26. In one example, as shown in FIGS. 19a and 19b, a desired spacing 246 between the apertures 214 is equal to or greater than the width 222 of the apertures 214. In this example, FIGS. 19c and 19d depict an undesired spacing 246 which is less than the width 222 of the apertures 214. In one example, it was recognized that adjusting the spacing 246 of the apertures 214 in the first zone 206 to be equal to or greater than the width 222 advantageously avoids a “net” appearance in the zonal topsheet 26 and ensures enough absorbent material is provided to isolate the skin from the wet layers of the first zone 206 below.

In one example, the apertures 214 formed in the zones of the zonal topsheet 26 are provided for absorbency, dryness, BM handling. In some examples, the apertures 214 are formed such that they are or look open or connected to the layers below. In an example, not only the width 222 of the apertures 214 is factored but also the density and depth of the apertures 214. In an example, a carded apertured 24 gsm Hospital TS from Yanjan® (code OS) is provided as one example with a desired aperture size/density/depth balance. A desired width and depth of the apertures formed in the zonal topsheet will now be discussed.

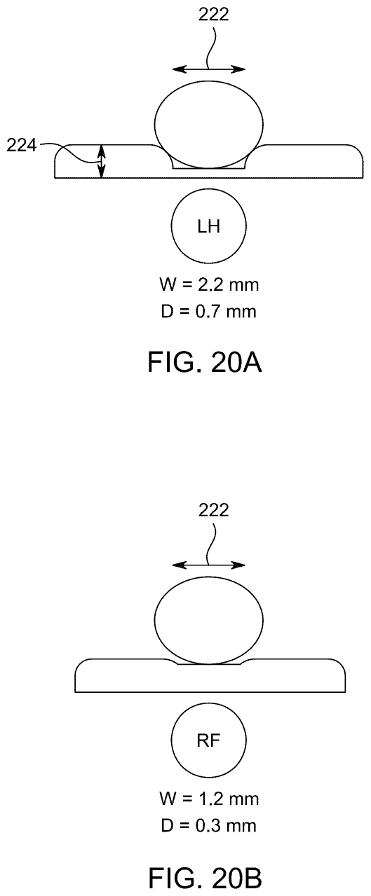

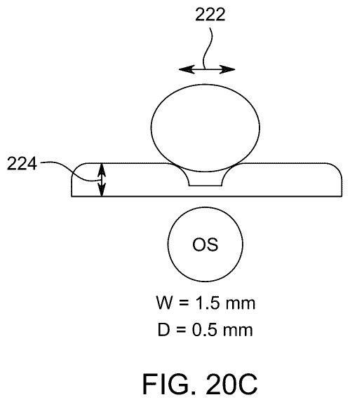

FIGS. 20a through 20c are plan views of an example of parameter values of apertures 214 formed in one or more zones of the zonal topsheet 26. In some examples, apertures 214 are formed in each zone of the zonal topsheet 26, but the values of the various characteristics (e.g. width, depth, density, etc.) may vary between the first zone 206 and the remaining zones of the zonal topsheet 26. Thus, in this example, none of the zones of the zonal topsheet 26 lack any apertures. In an example, the apertures 214 formed in the zonal topsheet have a width 222 that may be measured in a direction parallel to the central lateral axis 48. In one example, the value of the width 222 of the apertures 214 formed in the first zone 206 may be in a range between about 1 mm and about 2 mm. In this example, the value of the width 222 of the apertures 214 formed in the other zones 208, 209, 228, 230 may be less than the value of the width 222 of the apertures 214 formed in the first zone 206.

FIGS. 20a and 20b depict an undesired combination of width 222 and depth 224 of the apertures 214 formed in the first zone 206. FIG. 20c depicts a desired combination of width 222 and depth 224 of the apertures 214 formed in the first zone 206. In one example, the depth 224 of the apertures 214 formed in the first zone 206 may have a value equal to or greater than a fraction (e.g. one third) of the value of the aperture width 222. As shown in FIG. 20c, the value of the aperture depth 224 (e.g. 0.5 mm) is equal to or less than one third of the value of the aperture width 222 (e.g. 1.5 mm) and thus this combination of aperture width 222 and depth 224 is within a desired range for the apertures 214 formed in the first zone 206. In contrast with the desired aperture width and depth arrangement of FIG. 20c, the value of the aperture depth 224 of FIG. 20a (0.7 mm) is less than one-third the value of the aperture width 222 (e.g. 2.2 mm) and thus this is an undesired combination of aperture depth and width. Similarly, the value of the aperture depth 224 of FIG. 20b (e.g. 0.3 mm) is less than one-third the value of the aperture width 222 (e.g. 1.2 mm) and thus this is another undesired combination of aperture depth and width.

In yet another example, the value of the aperture width 222 of the apertures 214 formed in the first zone 206 cannot be too small or the apertures 214 will not provide sufficient absorption and cannot be too large or this will result in undesired skin contact with the absorbed waste in the lower layers of the absorbent article 10.

Similarly, in another example if the value of the aperture depth 224 results in apertures 214 that are too flat, this results in an undesired lack of separation between the skin and the absorbed waste in the lower layers of the absorbent article 10. Similarly, if the density of the apertures 214 is too high in the first zone 206, the topsheet 26 will not provide an adequate amount of material/substance to absorb the waste while also keeping the skin away from the absorbed waste in the lower layers of the absorbent article 10.

In an example, each zone of the zonal topsheet 26 may have some apertures 214 formed therein (e.g. with a non-zero depth 224 and width 222) and thus in this example none of the zones of the zonal topsheet 26 are entirely formed without apertures 214.

### Color Aspects of Zonal Topsheet

In some examples, the first zone 206 is visually discernable from the other zones of the zonal topsheet 26 based on having a different color than the other zones. In one example, the color difference between the first zone 206 and the other zones of the zonal topsheet includes at least a portion of the first zone 206 having a different L-a-b value than a portion of the one or more other zones of the zonal topsheet 26. The Lab Color Space will now be discussed herein, which is used to determine the different L-a-b value between the portion of the first zone 206 and the portion of the one or more other zones of the zonal topsheet 26.

The Lab Color Space (CIELAB color space): also referred to as L*a*b* is a color space defined by the International Commission on Illumination (CIE) in 1976. It expresses color as three values: L* for perceptual lightness, and a* and b* for the four unique colors of human vision: red, green, blue, and yellow. L* may range from 0 (black) to 100 (white); a* specifies redness-greenness and may range from negative values (green) to positive values (red); and b* specifies yellowness-blueness and may range from negative values (blue) to positive values (yellow). The CIELAB color space is device-independent, meaning that it is not tied to any device or display technology. This makes it a good choice for applications where color needs to be accurately represented on a variety of devices.

The color difference between two colors may be calculated by measuring the L*a*b* values for each color. The value of ΔE*ab, as specified in the following formula, is a measure of the perceived color difference between the two colors. The higher the value of ΔE*ab, the greater the perceived color difference.

$$Δ {E}^{*} ab = √ ( {( Δ {L}^{*} )}^{2} + {( Δ {a}^{*} )}^{2} + {( Δ {b}^{*} )}^{2} )$$

where ΔL*, Δa*, and Δb* are the differences in the L*, a*, and b* values between the two colors.

$$Δ {L}^{*} = {L}_{washed} - {L}_{i nitial}$$

$$Δ {a}^{*} = {a}_{washed} - {a}_{initial}$$

$$Δ {b}^{*} = {b}_{washed} - {b}_{i nitial}$$

### Data Supporting Unexpected Results

As disclosed herein, the improved zonal topsheet 26 was developed, where the area of the first zone 206 (e.g. absorbent zone) may be shrunk as compared with related art zonal topsheets. As shown in the examples of FIGS. 17b through 17g, the shape of the first zone 206 may be reshaped from a rectangular shaped zone to a modified zone with a narrowing width 212 towards a region (e.g. center) of the first zone 206. Thus, this first zone 206 of the improved zonal topsheet 26 may reduce the area of the absorbent zone from what was disclosed in related art zonal topsheets.

In yet another example, an area of the first zone 206 (e.g. absorbent zone) of the improved zonal topsheet 26 may be reduced from the area of absorbent zones in related art zonal topsheets, by limiting the width 212 and/or length 210 of the first zone 206 based on the structures (e.g. leg cuffs, solid excrement pocket, waistband, etc.) provided on the surface of the topsheet 26. As previously discussed, in some examples the width 212 of the first zone 206 is limited, based on the width W between the inboard edges 202, 204 of the leg cuffs 32a, 32b. As previously disclosed herein, this reduction of the area of the absorbent first zone 206 area was performed in order to ensure the different zones of the zonal topsheet 26 would remain visually discernable to the user and not be obscured by the leg cuffs 32a, 32b.

In spite of the above examples of where the area of the absorbent first zone 206 of the improved zonal topsheet 26 was reduced, as compared with related art topsheets, there was not an expected reduction in the amount of absorbed waste in the absorbent article. This is an unexpected result, since it was expected that reduction of the first absorbent zone 206 area would consequentially result in a reduction in the amount of absorbed waste at the first zone 206.

### Material Softness

Softness, texture (i.e., smoothness), and/or stiffness of a garment-facing surface of an outer cover material 40 may be measured by an Emtec Tissue Softness Analyzer, according to the Emtec Test herein. Tactile softness is measured as TS7. Texture/Smoothness is measured as TS750. Stiffness is measured as D. A portion of, or all of, garment-facing sides or surfaces 43 of the outer cover nonwoven materials 40 of the present disclosure may have a TS7 value in the range of about 1 dB V2 rms to about 8 dB V2 rms, about 1 dB V2 rms to about 6 dB V2 rms, about 2 dB V2 rms to about 4.5 dB V2 rms, or about 2 dB V2 rms to about 4.0 dB V2 rms. The portion of, or all of, the garment-facing surfaces of the outer cover materials of the present disclosure may also have a TS750 value in the range of about 2 dB V2 rms to about 30 dB V2 rms, about 6 dB V2 rms to about 30 dB V2 rms, about 6 dB V2 rms to about 20 dB V2 rms, about 6 dB V2 rms to about 15 dB V2 rms, about 6 dB V2 rms to about 12 dB V2 rms, or about 6.5 dB V2 rms to about 10 dB V2 rms. The portion of, or all of, the garment-facing surfaces of the outer cover materials of the present disclosure may also have a D value in the range of about 1 mm/N to about 10 mm/N, about 3 mm/N to about 8 mm/N, about 2 mm/N to about 6 mm/N, about 2 mm/N to about 4 mm/N, or about 3 mm/N to about 4 mm/N. All values are measured according to the Emtec Test herein.

### Emtec Test

The Emtec Test is performed on garment-facing surfaces of outer cover nonwoven materials. In this test, TS7, TS750, and D values are measured using an Emtec Tissue Softness Analyzer (“Emtec TSA”) (Emtec Electronic GmbH, Leipzig, Germany) interfaced with a computer running Emtec TSA software (version 3.19 or equivalent). The Emtec TSA includes a rotor with vertical blades which rotate on the test sample at a defined and calibrated rotational speed (set by manufacturer) and contact force of 100 mN. Contact between the vertical blades and the test sample creates vibrations both in the blades and in the test piece, and the resulting sound is recorded by a microphone within the instrument. The recorded sound file is then analyzed by the Emtec TSA software to determine TS7 and TS750 values. The D value is a measure of sample stiffness and is based on the vertical distance required for the contact force of the blades on test sample to be increased from 100 mN to 600 mN. The sample preparation, instrument operation, and testing procedures are performed according to the instrument manufacturer's specifications.

### Basis Weight Test

The basis weight of a nonwoven material of interest may be determined by several available techniques. A simple representative technique involves first excising that nonwoven from an absorbent article or other consumer product. The nonwoven is separated from any and all adjacent components (e.g. elastics) and layers. If nonwoven is adhesively bonded to elastics or to other nonwovens, films, or substrates, removal of the nonwoven material from other layers may be aided by the use of cryogenic spray (such as Cyto-Freeze, Control Company, Houston, Texas, or equivalent). If excess adhesive is present on the nonwoven is after excision, an appropriate solvent (e.g. THF, hexanes, etc.) may be used to remove residual adhesive present. Five like specimens of nonwoven are excised from the corresponding location in each of five like absorbent articles or other consumer products.

Each of the five nonwoven specimens is cut to produce a specimen portion with precisely known area for subsequent mass determination. (Only pristine nonwoven, undamaged during excision, can be used henceforth.) This is easily facilitated via the use of a steel rule die punch of known area (such as a circle with diameter 10 mm and area 78.5 mm²). In order to increase measurement precision, multiple punched areas from a single specimen nonwoven may be produced and then weighed together as a specimen portion. If, for example, three punches of a circle 10 mm in diameter were taken from the overall specimen, the area of the resulting specimen portion used subsequently would be 78.5 mm²×3=236 mm².

An analytical four-place balance is used to determine the mass of each of the five specimen portions of precisely known area, and the mass of each is recorded to the nearest 0.0001 g. The basis weight of each specimen is then determined using the known area of each specimen portion, and the basis weight of each is recorded to the nearest 0.1 gram per square meter (gsm). Finally, the arithmetic mean of the five specimen basis weights is calculated and reported to the nearest integer in units of gsm as the basis weight of the nonwoven of interest.

### Sample Preparation

A test sample is prepared by cutting a square or circular portion of interest from the outer cover nonwoven material of an absorbent article. It is preferable that freeze spray is not used to remove the portion of the outer cover nonwoven material to be analyzed, though it is acceptable to use freeze spray in a distal region to aid in initiating the separation of layers. Test samples are cut to a length and width (diameter in the case of a circular sample) of no less than about 90 mm and no greater than about 120 mm to ensure the sample can be clamped into the TSA instrument properly. (If an absorbent article does not contain a sufficiently large area of the substrate of interest to extract a sample of the size specified above, it is acceptable to sample equivalent material from roll stock.) Test samples are selected to avoid unusually large creases or folds within the testing region. Six substantially similar replicate samples are prepared for testing.

All samples are equilibrated at TAPPI standard temperature and relative humidity conditions (23° C.±2 C.° and 50%±2%) for at least 2 hours prior to conducting the TSA testing, which is also conducted under TAPPI conditions.

### Testing Procedure

The instrument is calibrated according to the Emtec's instructions using the 1-point calibration method with the appropriate reference standards (so-called “ref.2 samples,” or equivalent, available from Emtec).

A test sample is mounted in the instrument with the surface of interest facing upward, and the test is performed according to the manufacturer's instructions. The software displays values for TS7, TS750, and D when the automated instrument testing routine is complete. TS7 and TS750 are each recorded to the nearest 0.01 dB V2 rms, and D is recorded to the nearest 0.01 mm/N. The test sample is then removed from the instrument and discarded. This testing procedure is performed individually on the corresponding garment-facing surfaces of interest of each of the six of the replicate samples.

The value of TS7, TS750, and D are each averaged (arithmetic mean) across the six sample replicates. The average values of TS7 and TS750 are reported to the nearest 0.01 dB V2 rms. The average value of D is reported to the nearest 0.01 mm/N.

### Surface Topography Test for Sdc and Vvc

In the Surface Topography Test, the areal surface topology of a nonwoven web sample surface is measured using optical profilometry. The 3D surface data are then processed and analyzed to extract the core height parameter Sdc and the core void volume parameter Vvc. All sample preparation and testing are performed in a conditioned room maintained at 23±2° C. and 50±2% relative humidity, and prepared samples are kept in this environment for at least 24 hours prior to measurement.

### Sample Preparation

To prepare a sample of nonwoven web material to be obtained from a finished absorbent article, the topsheet layer is removed from an absorbent article exposing the underlying absorbent layer. The topsheet layer is carefully removed in a manner that avoids distortion of the surface topography of the upper and lower surfaces of the material. A cryogenic spray (such as CYTO-FREEZE, Control Company, Houston Texas, or equivalent) may be used to facilitate clean separation of the topsheet material from the underlying absorbent layer. Samples of topsheet materials with any tears or residua of folds should not be used. Five replicate samples are prepared for testing.

### 3D Surface Image Acquisition

A three-dimensional (3D) surface topography image of the body facing surface of the topsheet sample is obtained using a structured-light 3D surface topography measurement system (a suitable surface topography measurement system is the Gocator 3210 commercially available from LMI Technologies Inc., Vancouver, Canada, or equivalent). These measurement systems are 3D snapshot sensors, meaning they capture an entire surface in 3D in a single snapshot. These sensors project several structured light patterns in a rapid sequence onto the target. The reflection of the pattern off the target is captured by two cameras. The sample target must remain stationary during the camera exposure of the light patterns. A Structured Light Modulator (SLM) produces a sequence of high resolution/high contrast light patterns using a blue LED. Two cameras capture the reflected light pattern from different viewing angles. The sensor can then use either stereo correlation or independent triangulation to generate 3D points from the light pattern. The result of the measurement is a 3D data set of surface height (defined as the Z-axis) versus displacement in the horizontal (XY) plane. This 3D data set can also be thought of as an image in which every pixel in the image has an associated XY displacement, and the value of the pixel is the recorded Z-axis height value. The system is set to produce a height image with a field of view of approximately 100×154 mm with an XY pixel resolution of approximately 86 microns.

The instrument is calibrated, installed, and operated according to the manufacturer's specifications. The sample is placed flat on the table beneath the camera with the zone identified for analysis within the field of view. The sample may be very gently pulled taut (not to stretching) along X and Y dimensions to flatten out any large-scale waviness, and weights may be placed on the sample outside of the measurement area to hold it taut. Using the instrument's control and acquisition software, a 3D surface topology image of the sample surface is collected by following the instrument manufacturer's recommended measurement procedures. The collected height image file is saved to an evaluation computer running a surface texture analysis software (a suitable texture analysis software is Mountains Map version 9.2, Digital Surf, Besançon, France, or equivalent).

### 3D Surface Image Analysis

Analysis of a surface height image is initiated by opening the image in the surface texture analysis software. A recommended filtration process is described in ISO 25178-2:2021. Accordingly, the following filtering procedure is performed on each image: 1) if the zone identified for analysis is smaller than the image field of view, select the largest rectangular region of interest that can fit within the sample or identified zone and crop the image to that size; 2) a Gaussian low pass S-filter with a nesting index (cut-off) of 300 microns to remove short scale components; 3) an F-operation of removing the least squares plane to level the surface; and 4) a Robust Gaussian high pass L-filter with a nesting index (cut-off) of 20 mm (ISO 16610-71) to remove long scale components. Both Gaussian filters are run utilizing end effect correction. This filtering procedure produces the S-L surface from which the areal surface texture parameters will be calculated.

The core height value, Sdc, as described in ISO 25178-2:2021, is derived from the Areal Material Ratio (Abbott-Firestone) curve, which is the cumulative curve of the surface height distribution histogram versus the range of surface heights. The core height value is the height difference between the material ratios Smr1 and Smr2 as read off the Areal Material Ratio curve. Smr1, set to 5%, is the material ratio which separates the protruding peaks from the core roughness region. Smr2, set to 95%, is the material ratio which separates the deep valleys from the core roughness region. Record the surface height Sdc value to the nearest 0.01 mm. Average together the five replicate Sdc values and report to the nearest 0.01 mm.

The Surface Void Volume measurement is based on the Core Void Volume (Vvc) parameter which is described in ISO 25178-2:2012. The parameter Vvc is derived from the Areal Material Ratio (Abbott-Firestone) curve described in the ISO 13565-2:1996 standard extrapolated to surfaces, it is the cumulative curve of the surface height distribution histogram versus the range of surface heights. A material ratio is the ratio, given as a %, of the intersecting area of a plane passing through the surface at a given height to the cross-sectional area of the evaluation region. Vvc is the difference in void volume between p and q material ratios. The Surface Void Volume is the volume of void space above the surface of the sample between the height corresponding to a material ratio value of 5% to the material ratio of 95%, which is the Vvc parameter calculated with a p value of 5% and q value of 95%. The units of Surface Void Volume are mm³/mm². Record the Vvc value to the nearest 0.001 mm³/mm². Average together the five replicate Vvc values and report to the nearest 0.001 mm³/mm².

### EXAMPLES

The following examples are put forth to provide those of ordinary skill in the art with a complete disclosure and description of how to perform the methods, how to make, and how to use various aspects disclosed and claimed herein. Efforts have been made to ensure accuracy with respect to numbers, but some errors and deviations should be accounted for. The purpose of the following examples is not to limit the scope of the various embodiments, but merely to provide examples illustrating specific aspects.

In all examples, basis weight was measured according to the Basis Weight Test Method provided herein and is reported in grams per square meter (gsm); the average core height of the main features (Sdc) was measured according to the Surface Topography Test for Sdc and Vvc provided herein and is reported in millimeters (mm); and, finally, the average core volume of the main features (Vvc) was measured according to the Surface Topography Test for Sdc and Vvc provided herein and is reported in cubic millimeters per square millimeter (mm³/mm²).

For clarity, it is noted that in surface texture analysis, Sdc is a parameter that represents the average height of the main features (peaks and valleys) of a surface after filtering out smaller irregularities. It helps describe the overall texture of the surface, indicating its roughness or smoothness. Vvc is a parameter that measures the volume of the voids (or gaps) in the core part of a surface. It essentially quantifies how much space is present below the average height of the surface features after filtering out smaller details. The higher the Vvc, the more space is present between the peaks.

### Example 1

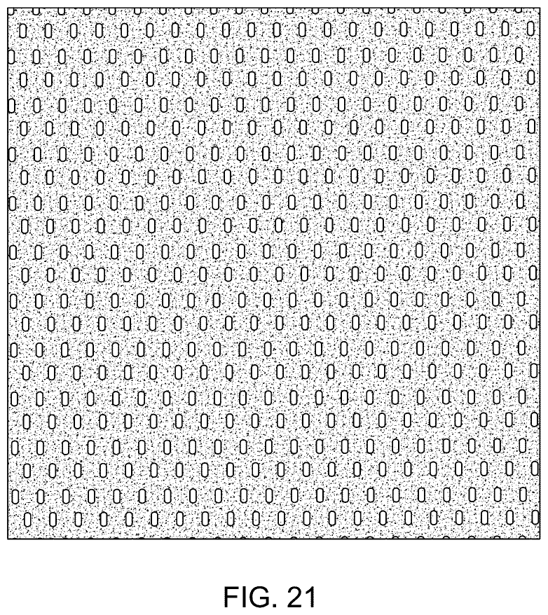

A purpose of this example is to demonstrate test results for a carded airthrough bonded apertured non-woven with PE/PET bicomponent fibers having a BW of 23 gsm, an Sdc parameter of 1.013 mm and a Vvc parameter of 0.288 mm³/mm². Such pattern and topography would be preferably located in the second zone. The results are summarized in Table 1. FIG. 21 provides a photograph of the final non-woven produced according to this example.

### Example 2

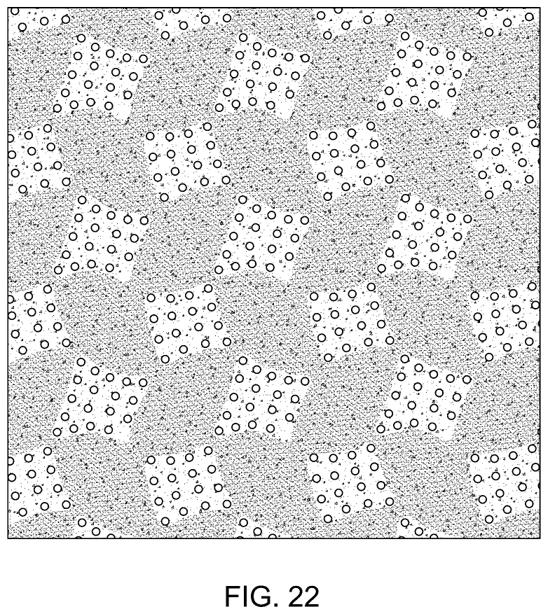

A purpose of this example is to demonstrate test results for a carded airthrough bonded apertured non-woven with PE/PET bicomponent fibers having a BW of 67 gsm, an Sdc parameter of 0.599 mm and a Vvc parameter of 0.1741 mm³/mm². Such pattern and topography would be preferably located in the second zone. The results are summarized in Table 1. FIG. 22 provides a photograph of the final non-woven produced according to this example.

### Example 3

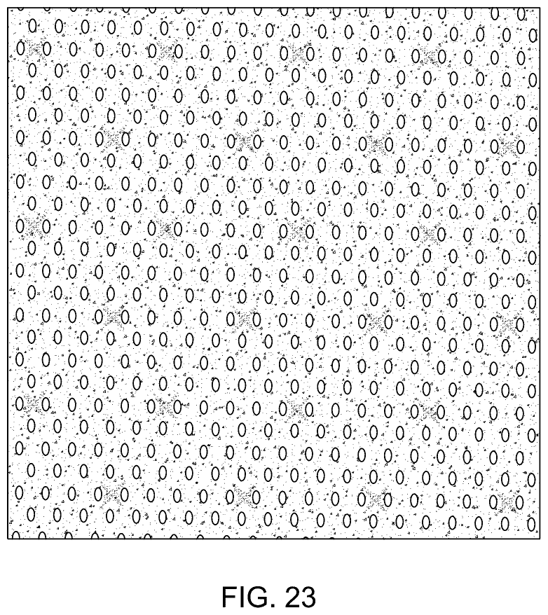

A purpose of this example is to demonstrate test results for a tri-layer composite material having a 23 gsm carded apertured airthrough bonded non-woven made out of PE/PET bicomponent fibers of 23 gsm on top, a 30 gsm carded airthrough bonded non-woven made out of PE/PET bicomponent fibers in the middle and a spunlace 42 gsm material at the bottom. All 3 layers are joined via ultrasonic bonding to achieve an overall basis weight of 95 gsm, an Sdc parameter of 1.292 mm and a Vvc parameter of 0.5073 mm³/mm². Such pattern and topography would be preferably located in the first zone. The results are summarized in Table 1. FIG. 23 provides a photograph of the final non-woven produced according to this example.

### Example 4

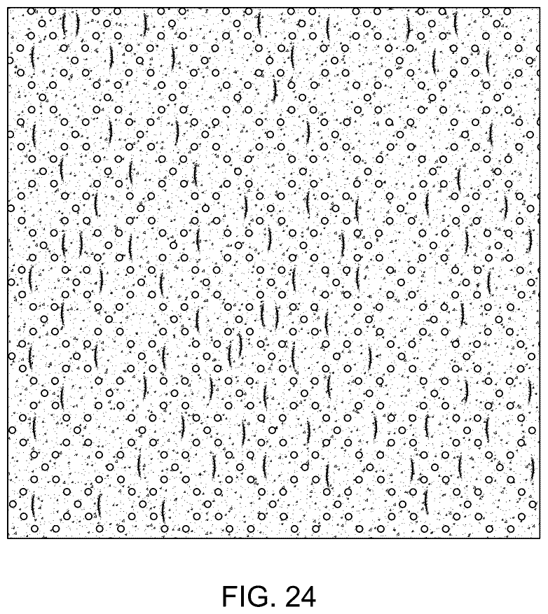

A purpose of this example is to demonstrate test results for a carded airthrough bonded apertured non-woven with PE/PET bicomponent fibers having a BW of 33 gsm, an Sdc parameter of 0.9813 mm and a Vvc parameter of 0.5547 mm³/mm². Such pattern and topography would be preferably located in the first zone. The results are summarized in Table 1. FIG. 24 provides a photograph of the final non-woven produced according to this example.

### Example 5

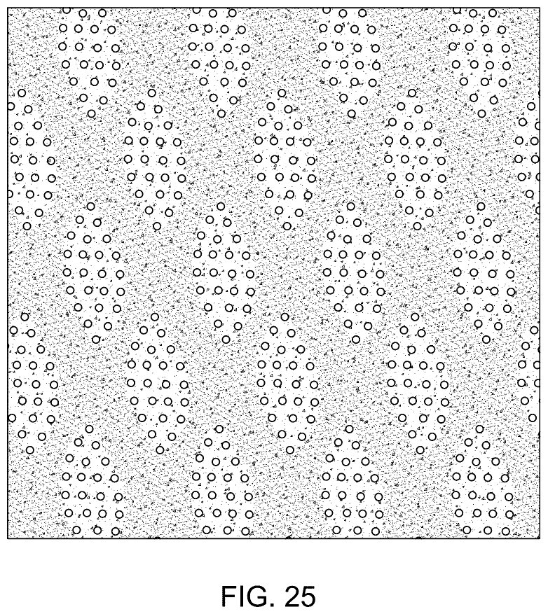

A purpose of this example is to demonstrate test results for a carded airthrough bonded apertured non-woven with PE/PET bicomponent fibers having a BW of 35 gsm, an Sdc parameter of 0.7291 mm and a Vvc parameter of 0.2948 mm³/mm². Such pattern and topography would be preferably located in the second zone. The results are summarized in Table 1. FIG. 25 provides a photograph of the final non-woven produced according to this example.

### Example 6

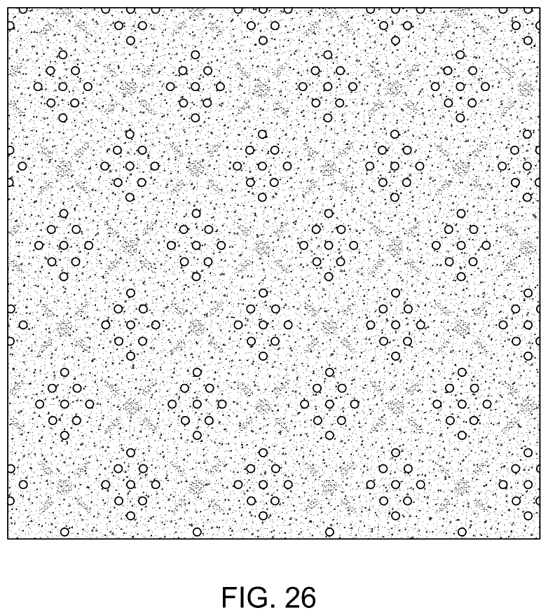

A purpose of this example is to demonstrate test results for a bi-layer carded airthrough bonded non-woven made out of PE/PET bicomponent fibers. Both layers are bonded together via ultrasonic bonding. The non-woven has a basis weight of 42 gsm and contains apertures. The non-woven has an Sdc parameter of 1.415 mm and a Vvc parameter of 0.5801 mm³/mm². Such pattern and topography would be preferably located in the first zone. The results are summarized in Table 1. FIG. 26 provides a photograph of the final non-woven produced according to this example.

### Example 7

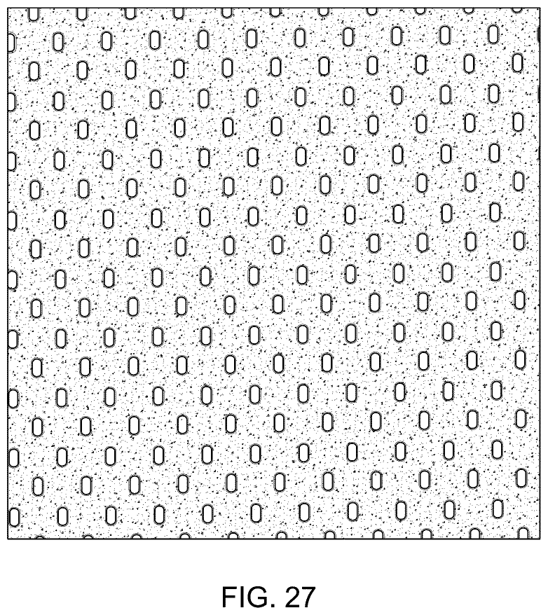

A purpose of this example is to demonstrate test results for a carded airthrough bonded apertured non-woven with PE/PET bicomponent fibers having a BW of 35 gsm, an Sdc parameter of 1.338 mm and a Vvc parameter of 0.3192 mm³/mm². Such pattern and topography would be preferably located in the first zone. The results are summarized in Table 1. FIG. 27 provides a photograph of the final non-woven produced according to this example.

### Example 8

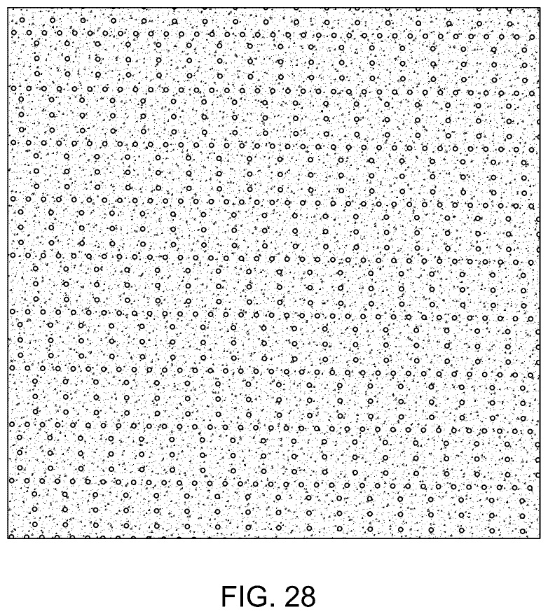

A purpose of this example is to demonstrate test results for a carded airthrough bonded apertured non-woven with PE/PET bicomponent fibers having a BW of 36 gsm, an Sdc parameter of 0.4924 mm and a Vvc parameter of 0.2382 mm³/mm². Such pattern and topography would be preferably located in the second zone. The results are summarized in Table 1. FIG. 28 provides a photograph of the final non-woven produced according to this example.

### Example 9

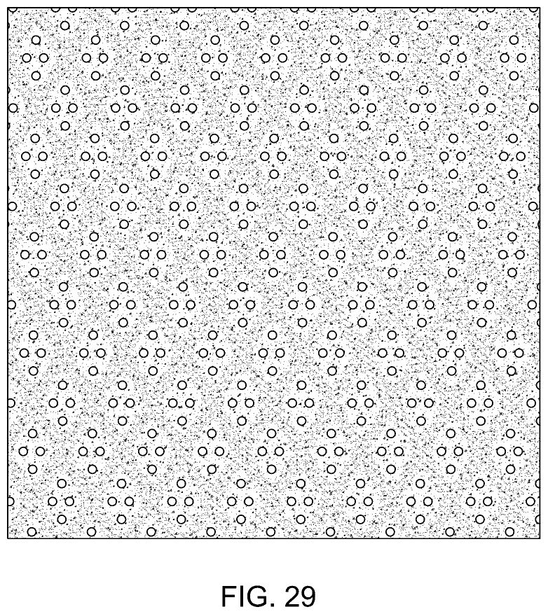

A purpose of this example is to demonstrate test results for a carded airthrough bonded apertured non-woven with PE/PET bicomponent fibers having a BW of 41 gsm, an Sdc parameter of 1.514 mm and a Vvc parameter of 0.6722 mm³/mm². Such pattern and topography would be preferably located in the first zone. The results are summarized in Table 1. FIG. 29 provides a photograph of the final non-woven produced according to this example.

### Example 10

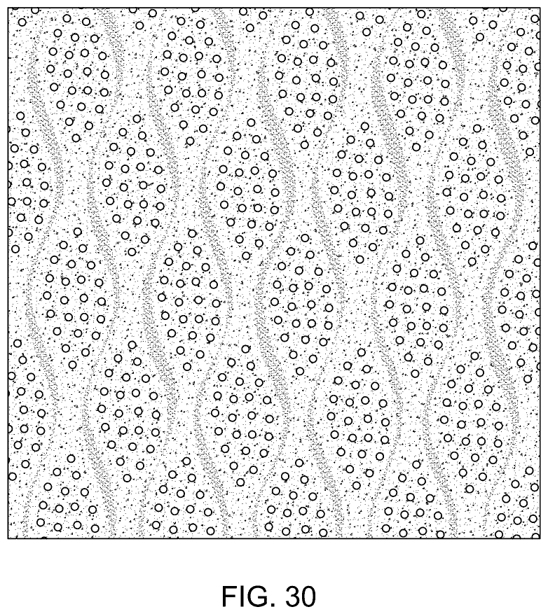

A purpose of this example is to demonstrate test results for a carded airthrough bonded apertured non-woven with PE/PET bicomponent fibers having a BW of 44 gsm, an Sdc parameter of 1.087 mm and a Vvc parameter of 0.4103 mm³/mm². Such pattern and topography would be preferably located in the first zone. The results are summarized in Table 1. FIG. 30 provides a photograph of the final non-woven produced according to this example.

TABLE 1

| Example | Basis weight (gsm) | Sdc (mm) | Vvc (mm³/mm²) |
|---|---|---|---|
| 1 | 23 | 1.013 | 0.288 |
| 2 | 67 | 0.599 | 0.1741 |
| 3 | 95 | 1.292 | 0.5073 |
| 4 | 33 | 0.9813 | 0.5547 |
| 5 | 35 | 0.7291 | 0.2948 |
| 6 | 42 | 1.415 | 0.5801 |
| 7 | 35 | 1.338 | 0.3192 |
| 8 | 36 | 0.4924 | 0.2382 |
| 9 | 41 | 1.514 | 0.6722 |
| 10 | 44 | 1.087 | 0.4103 |

The Sdc parameter may be in the range of about 0.4 mm to about 2 mm, or about 0.4 mm to about 1.5 mm, specifically reciting all 0.001 mm increments within the recited ranges and any ranges formed therein or thereby. The Vvc parameter may be in the range of about 0.1 mm³/mm² to about 1 mm³/mm², or about 0.1 mm³/mm² to about 0.8 mm³/mm², specifically reciting all 0.001 mm³/mm² increments within the recited ranges and any ranges formed therein or thereby.

### Numbered Recitations

1. An absorbent article comprising:
a first end edge;
a second end edge;
wherein the absorbent article has a longitudinal length, L, between the first end edge and the second end edge;
a first side edge;
a second side edge;
a central lateral axis extending in a transverse direction from the first side edge to the second side edge;
a central longitudinal axis extending in a longitudinal direction from the first end edge to the second end edge;
a first leg cuff comprising a first longitudinal inboard edge facing toward the central longitudinal axis;
a second leg cuff comprising a second longitudinal inboard edge facing toward the central longitudinal axis;
wherein a width between the first longitudinal inboard edge and the second longitudinal inboard edge, measured in a direction parallel to the central lateral axis, is W;
a liquid permeable topsheet having a wearer facing surface and a garment facing surface;
a liquid impermeable backsheet;
an absorbent core disposed at least partially between the topsheet and the backsheet;
the topsheet comprises at least two visually discernible zones comprising a first zone and a second zone, wherein the first zone has a first zone length that is 0.5 L to L, wherein the first zone has a width that is in the range of 0.7 W to 1.3 W, and wherein the first zone has a different core height value and/or a different core void volume than the second zone, according to the Surface Topography Test; and
wherein the first and second zones each comprise apertures and/or three-dimensional features on the wearer facing surface and/or the garment facing surface, wherein the three-dimensional features comprise morphological features that are at least partially concave or convex.

2. The absorbent article of Paragraph 1, wherein the width of the first zone is a minimum value of a first zone width along the length of the first zone.

3. The absorbent article of Paragraph 1, wherein the at least two visually discernible zones of the topsheet comprise both the apertures and the three-dimensional features.

4. The absorbent article of Paragraph 1, wherein the first zone has an area between 5,000 mm² and 50,000 mm², preferably between 10,000 mm² and 25,000 mm².

5. The absorbent article of Paragraph 1 or 2, wherein the topsheet is formed of a single material.

6. The absorbent article of Paragraph 1 or 2, wherein the topsheet comprises two materials, and wherein the two materials are joined by heat, chemistry, and/or mechanical force.

7. The absorbent article of any one of the preceding Paragraphs, wherein the first zone has a higher three-dimensionality than the second zone.

8. The absorbent article of any one of the preceding Paragraphs, wherein the first zone has a different permeability than the second zone. 9. The absorbent article of any one of the preceding Paragraphs, wherein the first zone has a lower softness than the second zone, as determined by the Emtec test.

10. The absorbent article of any one of the preceding Paragraphs, wherein at least a portion of the first zone has a different L-a-b value than at least a portion of the second zone.

11. The absorbent article of any one of the preceding Paragraphs, wherein the first zone comprises apertures having an aperture width in a direction parallel to the central lateral axis between about 1 mm and about 5 mm, preferably between about 1 mm and about 3 mm and a corresponding depth equal to or greater than one third of the aperture width.

12. The absorbent article of any one of the preceding Paragraphs, wherein the first zone comprises apertures having a larger dimension and/or three-dimensional features having a higher three-dimensionality than the second zone.

13. The absorbent article any one of the preceding Paragraphs, wherein a transition area between the first zone and the second zone comprises an arcuate portion and wherein the transition area is in the form of channels.

14. The absorbent article any one of the preceding Paragraphs, comprising a lotion on the topsheet.

15. The absorbent article of Paragraph 14, wherein the first zone has a first amount of lotion, wherein the second zone has a second amount of lotion, and wherein the first amount of lotion is different than the second amount of lotion.

16. The absorbent article of any one of the preceding Paragraphs, wherein the topsheet comprises a third zone.

17. The absorbent article of Paragraph 16, wherein the third zone is substantially similar to the second zone.

18. The absorbent article of Paragraph 16, wherein the first zone is positioned at least partially intermediate the second zone and the third zone.

19. The absorbent article of Paragraph 16, wherein the second zone is positioned proximate to the first end edge, and wherein the third zone is positioned proximate to the second end edge.

20. The absorbent article of Paragraph 16, wherein the second zone is positioned proximate to the first side edge, and wherein the third zone is positioned proximate to the second side edge.

21. The absorbent article of any one of Paragraphs 1-15, wherein the second zone is positioned proximate to the first end edge, comprising a third zone positioned proximate to the second end edge, comprising a fourth zone positioned proximate to the first side edge, comprising a fifth zone positioned proximate to the second side edge, wherein the first zone is positioned at least partially intermediate the second zone and the third zone, and wherein the first zone is positioned at least partially intermediate the fourth zone and the fifth zone.

22. The absorbent article of any one of the preceding Paragraphs, comprising a waistband, a waistcuff, or a BM pocket proximate to the first end edge and/or the second end edge.

23. The absorbent article of any one of the preceding Paragraphs, wherein the topsheet comprises spunbond fibers or carded fibers or a combination thereof.

24. The absorbent article of any one of the preceding Paragraphs, wherein the first zone has a different pattern as the second zone.

25. The absorbent article of any one of the preceding Paragraphs, wherein the absorbent article is a diaper comprising one or more of a pants diaper or a taped diaper.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the present disclosure. It is therefore intended to cover in the appended claims all such changes and modifications that are within the scope of this present disclosure.

What is claimed is:

1. An absorbent article comprising:
a first end edge;
a second end edge;
wherein the absorbent article has a longitudinal length, L, between the first end edge and the second end edge;
a first side edge;
a second side edge;
a central lateral axis extending in a transverse direction from the first side edge to the second side edge;
a central longitudinal axis extending in a longitudinal direction from the first end edge to the second end edge;
a first leg cuff comprising a first longitudinal inboard edge facing toward the central longitudinal axis;
a second leg cuff comprising a second longitudinal inboard edge facing toward the central longitudinal axis;
wherein a width between the first longitudinal inboard edge and the second longitudinal inboard edge, measured in a direction parallel to the central lateral axis, is W;
a liquid permeable topsheet having a wearer facing surface and a garment facing surface;
a liquid impermeable backsheet;
an absorbent core disposed at least partially between the topsheet and the backsheet;
the topsheet comprises at least two visually discernible zones comprising a first zone and a second zone, wherein the first zone has a first zone length that is 0.5 L to L, wherein the first zone has a width that is in the range of 0.7 W to 1.3 W, and wherein the first zone has a different core height value and/or a different core void volume than the second zone, according to the Surface Topography Test; and
wherein the first and second zones each comprise apertures and/or three-dimensional features on the wearer facing surface and/or the garment facing surface, wherein the three-dimensional features comprise morphological features that are at least partially concave or convex.

2. The absorbent article of claim 1, wherein the width of the first zone is a minimum value of a first zone width along the length of the first zone.

3. The absorbent article of claim 1, wherein the at least two visually discernible zones of the topsheet comprise both the apertures and the three-dimensional features.

4. The absorbent article of claim 1, wherein the first zone has an area between 5,000 mm² and 50,000 mm².

5. The absorbent article of claim 1, wherein the topsheet is formed of a single material.

6. The absorbent article of claim 1, wherein the topsheet comprises two materials, and wherein the two materials are joined by heat, chemistry, and/or mechanical force.

7. The absorbent article of claim 1, wherein the first zone has a higher three-dimensionality than the second zone.

8. The absorbent article of claim 1, wherein the first zone has a different permeability than the second zone.

9. The absorbent article of claim 1, wherein the first zone has a lower softness than the second zone, as determined by the Emtec test.

10. The absorbent article of claim 1, wherein at least a portion of the first zone has a different L-a-b value than at least a portion of the second zone.

11. The absorbent article of claim 1, wherein the first zone comprises apertures having an aperture width in a direction parallel to the central lateral axis between about 1 mm and about 5 mm and a corresponding depth equal to or greater than one third of the aperture width.

12. The absorbent article of claim 1, wherein the first zone comprises apertures having a larger dimension and/or three-dimensional features having a higher three-dimensionality than the second zone.

13. The absorbent article of claim 1, wherein a transition area between the first zone and the second zone comprises an arcuate portion and wherein the transition area is in the form of channels.

14. The absorbent article of claim 1, comprising a lotion on the topsheet.

15. The absorbent article of claim 14, wherein the first zone has a first amount of lotion, wherein the second zone has a second amount of lotion, and wherein the first amount of lotion is different than the second amount of lotion.

16. The absorbent article of claim 1, wherein the topsheet comprises a third zone.

17. The absorbent article of claim 16, wherein the third zone is substantially similar to the second zone.

18. The absorbent article of claim 16, wherein the first zone is positioned at least partially intermediate the second zone and the third zone.

19. The absorbent article of claim 16, wherein the second zone is positioned proximate to the first end edge, and wherein the third zone is positioned proximate to the second end edge.

20. The absorbent article of claim 16, wherein the second zone is positioned proximate to the first side edge, and wherein the third zone is positioned proximate to the second side edge.

21. The absorbent article of claim 1, wherein the second zone is positioned proximate to the first end edge, comprising a third zone positioned proximate to the second end edge, comprising a fourth zone positioned proximate to the first side edge, comprising a fifth zone positioned proximate to the second side edge, wherein the first zone is positioned at least partially intermediate the second zone and the third zone, and wherein the first zone is positioned at least partially intermediate the fourth zone and the fifth zone.

22. The absorbent article of claim 1, comprising a waistband, a waistcuff, or a BM pocket proximate to the first end edge and/or the second end edge.

23. The absorbent article of claim 1, wherein the topsheet comprises spunbond fibers or carded fibers or a combination thereof.

24. The absorbent article of claim 1, wherein the first zone has a different pattern than the second zone.
